(19) United States
(12) Patent Application Publication
(10) Pub. No.: US 2024/0342116 A1
(43) Pub. Date: Oct. 17, 2024

(54) TARGETING SOX10+ BONE MARROW GLIAL CELLS FOR TREATING MYELOFIBROSIS

(71) Applicant: The Trustees of Columbia University in the City of New York, New York, NY (US)

(72) Inventors: Lei Ding, Closter, NJ (US); Shawn M. Sarkaria, New York, NY (US); Junsong Zhou, New York, NY (US)

(21) Appl. No.: 18/629,157

(22) Filed: Apr. 8, 2024

Related U.S. Application Data
(60) Provisional application No. 63/458,743, filed on Apr. 12, 2023.

(51) Int. Cl. A61K 31/137; A61K 45/06

(52) U.S. Cl. CPC A61K 31/137; A61K 45/06

(57) ABSTRACT

Methods and compositions for treating a myelofibrosis with an agent that depletes, or inhibits proliferation of, Sox10+ glial cells in a subject and/or ErbB3+ glial cells in a subject.

### CROSS-REFERENCE TO RELATED APPLICATIONS

This application claims benefit of U.S. Provisional Application No. 63/458,743, filed Apr. 12, 2023, the contents of which are hereby incorporated by reference.

### GOVERNMENT SUPPORT CLAUSE

This invention was made with government support under HL155868 and HL153487 awarded by the National Institutes of Health. The government has certain rights in the invention.

### BACKGROUND OF THE INVENTION

Throughout this application, various publications are referenced, including by number. The disclosures of all publications mentioned in this application in their entireties are hereby incorporated by reference into this application in order to provide additional description of the art to which this invention pertains and of the features in the art which can be employed with this invention.

Alterations in tissue microenvironment contribute critically to the pathogenesis of many diseases. In hematopoietic malignancies, remodeling of the stromal compartment in the bone marrow niche is emerging as an important component promoting the propagation of mutant hematopoietic cells. This raises the possibility of targeting the niche to better treat hematopoietic malignancies.

Primary myelofibrosis (PMF) is a severe type of myeloproliferative neoplasm (MPN) characterized by megakaryocyte atypia, bone marrow fibrosis, and extramedullary hematopoiesis. Most MPNs arise from a single gain-of-function mutation, commonly in JAK2, MPL, or CALR, that causes aberrant activation of the MPL-JAK-STAT signaling pathway and dysregulated proliferation of myeloid cells 1. Like human disease, mouse models of MPNs display a range of phenotypes: JAK2V617F largely leads to polycythemia vera (PV) or essential thrombocythemia (ET) while THPO overexpression (TOE) or MPLW515L leads to lethal PMF 2-5. It is not entirely clear how similar mutations can bring about such a broad range of clinical features, although this diversity seems to be influenced by the mutant allele frequencies and cooperating genetic alterations 6. Nonetheless, bone marrow fibrosis accompanied by progressive hematopoietic dysfunction in PMF reflects profound alterations in the bone marrow microenvironment. However, our understanding of the stromal cellular changes that drive bone marrow remodeling in PMF remains incomplete.

The bone marrow niche under steady-state conditions has been studied extensively. Leptin receptor-positive (LepR+) perivascular mesenchymal stromal cells (MSCs) and endothelial cells have emerged as major niche constituents and important sources of hematopoietic stem cell (HSC) maintenance factors including SCF and CXCL12 7-10. LepR+ cells also have properties of mesenchymal stem/progenitor cells and play an important role in adult osteogenesis 10-12. Other less abundant stromal cells include periarteriolar pericytes and glial cells 13,14. Several recent reports of large-scale single-cell transcriptional profiling have confirmed the heterogeneous composition of the bone marrow stroma 15-17. How these heterogeneous bone marrow stromal cells contribute to PMF is not well understood.

### SUMMARY OF THE INVENTION

A method for treating a myelofibrosis in a subject comprising administering to the subject a pharmaceutical composition comprising an agent that depletes, or inhibits proliferation of, Sox10+ glial cells in a subject and/or ErbB3+ glial cells in a subject, so as to thereby treat the myelofibrosis.

A bone marrow-targeting therapeutic composition comprising (i) an agent that depletes, or inhibits proliferation of, Sox10+ glial cells in a subject and/or ErbB3+ glial cells and (ii) a molecular entity that targets bone marrow.

A method for treating a myelofibrosis in a subject comprising administering to the subject an agent that depletes, or inhibits proliferation of, ErbB3+ glial cells in a subject, and a Janus kinase inhibitor, so as to thereby treat the myelofibrosis.

### BRIEF DESCRIPTION OF THE DRAWINGS

FIGS. 1A-1O: Gli1- and LepR-lineage cells occupy largely distinct regions within long bones. 1A-1E, Representative confocal images showing widespread distribution of LepR+ cells throughout the bone marrow. 1B-1E are magnified images corresponding to the regions outlined by white boxes in panel A (n=4). 1F-1J, Representative confocal images showing Gli1+ cells are concentrated in and around the growth plate (primary spongiosa, growth plate chondrocytes), with sparse presence close to the endosteal surface in the diaphysis region. Few, if any, Gli1+ cells are observed in the central bone marrow. 1G-1J are magnified images corresponding to the regions outlined by white boxes in panel 1F (n=10, 1-month post-tamoxifen). 1K, Quantification of LepR- and Gli1-lineage cells in the bone marrow by confocal microscopy. tdTomato+ cells were enumerated and averaged across 5 randomly chosen high-power fields within the central marrow (i.e. middle area of the femoral diaphysis excluding the endosteal lining) of 3 independent mice. 1L, Flow cytometric quantification of LepR- and Gli1-lineage stromal cells in the bone marrow (n=3 for Lepr-Cre, n=5 for Gli1-CreER). 1M, qPCR analysis showing that Gli1-lineage cells express higher levels of osteolineage genes than LepR-lineage cells (n=3 for LepR+, n=4 for Gli1+). 1N, A representative confocal image of bone marrow section from Gli1-CreER; loxp-tdTomato mice showing close approximation of tdTomato+ cells and osteopontin (n=2). 1O, A representative confocal image of bone marrow section from Gli1-CreER; loxp-tdTomato; Col1a1-GFP mice showing tdTomato+ cells are Col1a1-GFP+ (n=3). Scale bars are 500 μm for panels 1A and 1F and 100 μm for all other panels. Data present mean±SD. Unpaired Student's t-tests were used to assess statistical significance. *p<0.05, ***p<0.001.

FIGS. 2A-2N: Gli1-lineage cells represent a small fraction of myofibroblasts in the TOE PMF model. 2A, Experimental schema of Gli1-CreER lineage tracing in TOE PMF model. 2B and 2C, Representative bone marrow histology changes in TOE PMF model as revealed by H&E (atypical megakaryocytes indicated by arrowheads, osteosclerosis indicated by paucicellular eosinophilic trabeculae) and reticulin staining (dense fibrosis). 2D, Local expansion of Gli1-lineage cells is present near trabecular bone regions but absent from the central marrow cavity in TOE mice, even though abnormal TOE hematopoietic cells (GFP+) are distributed throughout the bone marrow (n=12). 2E-2G, Confocal images show that Gli1-lineage cells are largely absent from the diaphyseal bone marrow even though abundant Col1a1-GFP+ myofibroblasts are present (E-F) in TOE PMF mice. In contrast, LepR-lineage cells give rise to the majority of Col1a1-GFP+ myofibroblasts in the central marrow cavity (G, high degree of GFP and tdTomato overlap). Bottom images show magnified regions corresponding to white boxes in the top images. 2H and 2I, Significant upregulation of stromal cell Col1a1-GFP in TOE PMF mice as shown by histogram shift and mean fluorescent intensity (MFI) quantification of flow cytometry data (n=3). 2J and 2K, Representative flow cytometry plots showing that the bone marrow has far fewer Gli1+ stromal cells compared with LepR+ stromal cells in TOE PMF mice (n=10 for TOE Gli1-CreER; loxp-tdTomato; n=4 for TOE Lepr-Cre; loxp-tdTomato). 2L-2N, Representative flow cytometry plots and summary statistics showing the fraction of Col1a1-GFP+ myofibroblasts that are tdTomato positive after PMF induction. LepR-lineage cells account for an average of 84%, while Gli1-lineage cells account for around 7% of all Col1a1-GFP+ cells in TOE PMF mice (n=3 for each genotype). Scale bars are 500 μm for whole bone images and 100 μm for magnified images in D-G. Data presented as mean±SD. Unpaired Student's t-tests were used to assess statistical significance. **p<0.01, ***p<0.001.

FIGS. 3A-3Q: Gli1 deletion from either stromal or hematopoietic cells does not modify hallmark features of PMF. 3A, Schema of transplantation of wildtype bone marrow cells transduced with TOE virus into Gli1 wildtype (Gli1+/+), hemizygous (Gli1+/−) or homozygous knockout (Gli1−/−) recipient mice. 3B and 3C, Representative images showing prominent splenomegaly in Gli1−/− and Gli1+/− recipient mice transplanted with TOE bone marrow cells versus empty vector control bone marrow cells. Stromal loss of Gli1 does not significantly impact splenomegaly in PMF mice (n=5 for Gli1+/+, n=6 for Gli1+/−, n=8 for Gli1−/−). 3D-3F, Gli1−/− recipient mice show similar degrees of pale bone, anemia, and thrombocytosis compared with Gli1+/+ recipient mice in PMF (n=3 for Gli1+/+ and n=4 for Gli1−/−). 3G-3K, Gli1−/− recipient mice have similar bone marrow cellularity, HSC frequency, spleen cellularity, and hematopoietic progenitor frequency compared with Gli1+/+ and Gli1+/− recipient mice in PMF (n=4 for Gli1+/+, n=5 for Gli1+/−, n=8 for Gli1−/−). 3L, Representative images of reticulin staining demonstrate similar degrees of bone marrow fibrosis in Gli1+/+, Gli1+/− or Gli1−/− recipient mice after PMF induction. 3M, Schema of experimental design involving transplantation of Gli1+/+ or Gli1−/− donor bone marrow cells transduced with Tpo into Gli1+/+ recipient mice to generate a PMF model with Gli1 deletion from the hematopoietic compartment. 3N, Flow cytometry plots showing similar myeloproliferation when using either Gli1+/+ or Gli1−/− donor bone marrow cells. 3O-3Q, Deletion of Gli1 from hematopoietic cells does not impact bone marrow HSC frequency, spleen hematopoietic progenitor frequency, or bone marrow fibrosis in PMF (n=3 for Gli1+/+ donor, n=5 for Gli1−/− donor). Data presented as mean±SD. Unpaired Student's t-tests were used to assess statistical significance. Scale bars represent 100 μm.

FIGS. 4A-4S: LepR-lineage cells are the major source of bone marrow myofibroblasts in a clinically relevant MPLW515L PMF model. 4A, Schema of MPLW515L PMF model for lineage tracing. 4B and 4C, Significantly elevated white blood cell and platelet counts in the MPLW515L PMF model (n=3 for controls and n=4 for MPLW515L). 4D, Flow cytometry plots showing bone marrow myeloproliferation in MPLW515L PMF model. FSC, forward scatter; SSC, side scatter. 4E and 4F, Prominent splenomegaly and hepatomegaly in MPLW515L PMF model (n=3 for controls and n=4 for MPLW515L). 4G and 4H, Representative histology images showing effacement of normal splenic architecture and extensive reticulin bone marrow fibrosis in MPLW515L PMF model. 4I and 4J, Compared with empty vector control mice, MPLW515L-induced PMF mice exhibit a significant expansion of LepR-lineage cells, which are the major source of Col1a1-GFP-expressing myofibroblasts throughout the bone marrow in Lepr-Cre; loxp-tdTomato; Col1a1-GFP mice. 4K, Flow cytometry analysis demonstrates a significant expansion of tdTomato+ LepR-lineage cells in MPLW515L PMF model (n=3 for controls and n=4 for MPLW515L). 4L, Modest expansion of Gli1-lineage cells near trabecular and endosteal regions but not within the central marrow cavity, where abundant Col1a1-GFP+ myofibroblasts are present in Gli1-CreER; loxp-tdTomato; Col1a1-GFP recipient mice transplanted with MPLW515L bone marrow. 4M, LepR-lineage cells account for 88% while Gli1-lineage cells account for only 4% of Col1a1-GFP+ myofibroblasts in MPLW515L PMF mice (n=3 for each genotype). 4N-4S, FACS-sorted LepR-lineage cells significantly downregulate HSC niche factors as well as Lepr, and upregulate fibrosis genes in the MPLW515L PMF model (n=3 for controls and n=5 for MPLW515L). Data presented as mean±SD. Unpaired Student's t-tests were used to assess statistical significance. *p<0.05, **p<0.01, ***p<0.001. Scale bars represent 500 μm in whole femur images and 100 μm in magnified images of 4I-4K.

FIGS. 5A-5J: Combined lineage-tracing and scRNA-seq identifies LepR+ mesenchymal stromal cells as the major cellular driver of bone marrow fibrosis. 5A, Schema of scRNA-seq analysis on stromal cells isolation from Lepr-Cre; loxp-tdTomato recipient mice. 5B and 5C, tSNE plots reveal a total of three distinct stromal lineage populations: LepR+ MSCs, endothelial cells, and glial cells. Within the LepR+ MSCs, 6 subclusters can be observed, including Lepr1-4, Lepr cycling, and pericytes. 5D, Cluster distribution of stromal cells within the bone marrow of control and PMF mice. 5E, Significant reduction of Lepr2 but increase of Lepr3, Lepr4, pericytes, and glial cells in PMF bone marrow compared with control. Error bars represent 95% confidence intervals of binomial fit mean using condition (control or disease) as the predictive factor. Four independent scRNA-seq experiments were performed for two control and four PMF mice. 5F and 5G, tdTomato+ MSCs are the major Col1a1 and Col3a1 producers in the bone marrow of Lepr-Cre; loxp-tdTomato mice. Note that pericytes but not glial cells are tdTomato+ (derived from LepR-lineage cells). 5H, Dot plot shows upregulation of extracellular matrix and matricellular genes in PMF compared to control. The Lepr3 population produces higher levels of ECM proteins implicated in osteogenesis whereas the Lepr4 population produces more matricellular proteins known to play a role in ECM remodeling. Circle size represents the percentage of cells expressing a given gene in a given cluster (0-100%) and color scale denotes the normalized level of gene expression. 5I, RNA velocity analysis showing that Lepr+ MSCs commit to three major cellular outputs in PMF: L1-osteolineage (Lepr3); L2-extracellular matrix remodeling (Lepr4); and L3-Acta2 (SMA)+ to pericytes through Lepr2. 5J, Expression changes of key markers in MSCs over pseudotime along the three major cell lineage fates identified in panel 5I. Lines indicate average gene expression over pseudotime for control (solid lines) and PMF (dashed lines) cells.

FIGS. 6A-6K: PMF bone marrow is a transformed cellular ecosystem with profound changes in endothelial and glial cells and their interactions with MSCs. 6A, Endothelial cells from PMF bone marrow downregulate sinusoidal and upregulate arteriolar markers. 6B, Endothelial cells upregulate the expression of CD31 (PECAM1) and show increased longitude branching in the PMF bone marrow. Quantification of vascular area is shown (n=3 for controls and n=5 for PMF). 6C, Sox10+ glial cells specifically express Gfap. 6D, Sox10+ glial cells express high levels of Postn and Itgb8 in the PMF bone marrow. 6E, Sox10-lineage cells expand in the PMF bone marrow of Sox10-CreER; loxp-tdTomato mice. Bone marrow endothelial cells were stained with an anti-CD31 antibody (in green). Arrowheads point to glial cells. Quantification of Sox10+ glial cells was based on 3 representative bone marrow images from each mouse (n=3 mice for each condition). 6F and 6G, Steady-state cell-cell communication between stromal cells is dominated by endothelial and MSC interactions. Pericyte and glial cell involvement is minimal given their scarcity (F). PMF bone marrow is characterized by a dramatic increase in cell-cell communication within the stromal cell compartment (G). Numbers indicate total interactions. 6H, Cell-cell communication pathways significantly enriched among bone marrow stromal cells isolated from PMF mice compared to controls. Teal color represents relative information flow in control cells whereas pink color represents relative information flow in PMF cells for each respective signaling network. 6I, Expression of Notch ligands and receptors in bone marrow stromal cells from control (teal) and PMF (pink) mice. 6J, Inferred intercellular communication network of Notch signaling in PMF showing pericytes as the major cell type sending Notch signals. 6K, Relative contribution of each ligand-receptor pair to the overall communication network of Notch signaling in PMF. Jag1 is the dominant Notch ligand among stromal cells in PMF and primarily derives from pericytes. Data presented as mean±SD. Unpaired Student's t-tests were used to assess statistical significance. Scale bars represent 100 μm in 6B and 6E.

FIGS. 7A-7N: Ablation of bone marrow glial cells ameliorates PMF pathology. 7A, Schema of bone marrow glial cell ablation with 6-OHDA in the MPLW515L PMF model. 7B, Levels of active TGF-β in the serum and the bone marrow fluid of MPLW515L PMF mice treated with 6-OHDA and vehicle (n=7 for controls and n=12 for 6-OHDA). 7C, Treatment of MPLW515L PMF mice with 6-OHDA leads to an increase in bone marrow cellularity (n=6 for controls and n=5 for 6-OHDA). 7D and 7E, Treatment of MPLW515L PMF mice with 6-OHDA leads to decreased sizes and weight of the spleen and liver (n=6 for controls and n=5 for 6-OHDA). 7F, MPLW515L PMF mice treated with 6-OHDA have ameliorated bone marrow fibrosis. 7G, Schema of bone marrow glial cell ablation in the Sox10-CreER; loxp-iDTR MPLW515L PMF model. 7H, Levels of active TGF-β in the serum and bone marrow fluid of Sox10-CreER; loxp-iDTR MPLW515L PMF model (n=13 for control and n=6 for DT-treated mice). 7I to 7L, DT-treated Sox10-CreER; loxp-iDTR MPLW515L PMF mice have an increase in bone marrow cellularity, similar HSC and hematopoietic progenitor frequencies, a decrease in spleen weight and a decrease in liver weight compared with controls (n=9 for control and n=6 for DT-treated mice for 7I, 7K, 7L; n=8 for control and n=6 for DT-treated mice for 7J). 7M, DT-treated Sox10-CreER; loxp-iDTR MPLW515L PMF mice have ameliorated bone marrow fibrosis (n=6 for control and n=5 for DT-treated mice). 7N, qPCR analysis showing the expression levels of Cxcl12 and Scf in bone marrow MSCs from DT-treated Sox10-CreER; loxp-iDTR and control mice in PMF (n=7 for controls and n=5 for Sox10-CreER; loxp-iDTR mice). Data presented as mean±SEM. Student's t-tests were used in B-E, H-L, and N. Mann-Whitney tests were used in 7F and 7M. Scale bars represent 50 μm in F and M. NS, not significant; *p<0.05, **p<0.01.

### DETAILED DESCRIPTION OF THE INVENTION

A method for treating a myelofibrosis in a subject comprising administering to the subject a pharmaceutical composition comprising an agent that depletes, or inhibits proliferation of, Sox10+ glial cells in a subject and/or ErbB3+ glial cells in a subject, so as to thereby treat the myelofibrosis.

In embodiments, the Sox10+ glial cells are not surgically ablated from the subject.

In embodiments, the agent is a small molecule.

In embodiments, the agent comprises 6-hydroxydopamine.

In embodiments, the pharmaceutical composition comprises a conjugate of 6-hydroxydopamine.

In embodiments, the agent is a cytotoxic agent which targets ErbB3 or an agent which blocks ErbB3.

In embodiments, the agent comprises an anti-ErbB3 antibody or ErbB3-binding fragment thereof.

In embodiments, the method does not evoke body weight loss in the subject.

In embodiments, the agent selectively depletes, or selectively inhibits proliferation of, Sox10+ glial cells over other glial cell types.

In embodiments, the agent does not cross a blood-brain barrier in the subject.

In embodiments, the method effects depletion of, or inhibition of proliferation of, Sox10+ glial cells in bone marrow of the subject.

In embodiments, the methods further comprise diagnosing the subject, or having the subject diagnosed, as having a myelofibrosis prior to treatment.

In embodiments, the subject is diagnosed on the basis of a bone marrow biopsy.

In embodiments, the subject is diagnosed with fibrosis grade 2 or 3.

In embodiments, the subject is or has been diagnosed with having a myelofibrosis which exhibits expanded Sox10+ glial cells in bone marrow of the subject.

In embodiments, the agent is not a Janus kinase (JAK) inhibitor.

In embodiments, the agent is administered systemically to the subject.

In embodiments, the agent is administered locally into bone marrow of the subject.

In embodiments, the myelofibrosis is a primary myelofibrosis.

In embodiments, the agent is an anti-ErbB3 antibody, or ErbB3-binding fragment thereof, and a JAK inhibitor is additionally administered to the subject.

In embodiments, the agent comprises AZD8931.

In embodiments, a JAK inhibitor is additionally administered to the subject.

A method for treating a myelofibrosis in a subject comprising administering to the subject an agent that depletes, or inhibits proliferation of, ErbB3+ glial cells in a subject, and a Janus kinase inhibitor, so as to thereby treat the myelofibrosis.

A method for treating a myelofibrosis in a subject comprising administering to the subject an agent that depletes, or inhibits proliferation of, Sox10+ glial cells in a subject, and a Janus kinase inhibitor, so as to thereby treat the myelofibrosis.

A bone marrow-targeting therapeutic composition comprising (i) an agent that depletes, or inhibits proliferation of, Sox10+ glial cells in a subject and/or ErbB3+ glial cells and (ii) a molecular entity that targets bone marrow.

In embodiments, the bone marrow-targeting therapeutic composition targets human bone marrow.

In embodiments, the bone marrow-targeting therapeutic composition comprises an agent that depletes Sox10+ glial cells which agent comprises 6-hydroxydopamine.

In embodiments, the bone marrow-targeting therapeutic composition comprises a conjugate of 6-hydroxydopamine.

In embodiments, the bone marrow-targeting therapeutic composition comprises a cytotoxic agent which targets ErbB3 or an agent which blocks ErbB3.

In embodiments, the agent comprises an anti-ErbB3 antibody or ErbB3-binding fragment thereof.

In embodiments, the molecular entity that targets bone marrow comprises a bisphosphonate, a tetracycline, an oligopeptide, or an aptamer.

In embodiments, the bone marrow-targeting therapeutic composition comprises a lipid nanoparticle (LNP) with the agent that depletes Sox10+ glial cells as a payload and the molecular entity that targets bone marrow is on an outside surface of the LNP.

In embodiments, the molecular entity that targets bone marrow is an antibody directed against a Sox10+ glial cell surface marker.

Janus kinase (JAK) inhibitors as contemplated in the invention herein are known in the art. See, e.g., Shawky et al. Pharmaceutics. 2022 May; 14(5): 1001, e.g., at www.ncbi.nlm.nih.gov/pmc/articles/PMC9146299/, hereby incorporated by reference in its entirety. JAK inhibitors include abrocitinib, baricitinib, delgocitinib, fedratinib, filgotinib, oclacitinib, pacritinib, peficitinib, ruxolitinib, tofacitinib, and upadacitinib.

In some embodiments the JAK inhibitor is administered to a human subject at about 5 mg to 500 mg per day. For example, one of at about 5 mg, at about 10 mg, at about 15 mg, at about 20 mg, at about 25 mg, at about 30 mg, at about 35 mg, at about 40 mg, at about 45 mg, at about 50 mg, at about 55 mg, at about 60 mg, at about 70 mg, at about 80 mg, at about 90 mg, at about 100 mg, at about 150 mg, at about 200 mg, at about 250 mg, at about 300 mg, at about 350 mg, at about 400 mg, at about 450 mg, or at about 500 mg.

Examples of anti-ErbB3 antibodies/fragments can be found in Gaborit et al., Hum. Vaccin. Immunother. 2016 March; 12(3): 576-592, e.g., at www.ncbi.nlm.nih.gov/pmc/articles/PMC4964743/, which is hereby incorporated by reference in its entirety. Examples include Elgemtumab (LJM716), Lumretuzumab (RG7116) and KTN3379. Examples of inhibitors of ErbB3 which are small molecules are known, for example, AZD8931.

In some embodiments the cytotoxic agent which targets ErbB3 or an agent which blocks ErbB3, for example AZD8931, is administered to a human subject either once daily or twice daily. In some embodiments the anti-Erbb agent is administered once daily or twice daily at a dose of 5 mg, 10 mg, 20 mg, 40 mg, 80 mg, 100 mg, 120 mg, 140 mg, 160 mg, or 180 mg. For example, in an embodiment the subject is administered a 5 mg, 10 mg, 20 mg, 40 mg, 80 mg, 100 mg, 120 mg, 140 mg or 160 mg bd dose.

In some embodiments the cytotoxic agent which targets ErbB3 or an agent which blocks ErbB3, for example AZD8931, is administered to a human subject at about 5 mg to 500 mg per day. For example, one of at about 5 mg, at about 10 mg, at about 15 mg, at about 20 mg, at about 25 mg, at about 30 mg, at about 35 mg, at about 40 mg, at about 45 mg, at about 50 mg, at about 55 mg, at about 60 mg, at about 70 mg, at about 80 mg, at about 90 mg, at about 100 mg, at about 150 mg, at about 200 mg, at about 250 mg, at about 300 mg, at about 350 mg, at about 400 mg, at about 450 mg, or at about 500 mg.

The agents or compounds, when small molecules, used in the method of the present invention can be in a salt form. As used herein, a “salt” is a salt of the compounds which has been modified by making acid or base salts of the compounds. The salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The compounds of the present invention can, in embodiments, also form salts with basic amino acids such a lysine, arginine, etc. and with basic sugars such as N-methylglucamine, 2-amino-2-deoxyglucose, etc. and any other physiologically non-toxic basic substance.

The agents or compounds, when antibodies or antibody fragments, used in the method of the present invention can be in any form that antibodies or antibody fragments are administered to humans. For example, see Awwad et al, Pharmaceutics. 2018 September; 10(3): 83, e.g., at https://www.ncbi.nlm.nih.gov/pmc/articles/PMC6161251/, which is hereby incorporated by reference in its entirety.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, into cerebral parenchyma or intraparenchchymally.

The compounds used in the method of the present invention may be administered in various forms, including those detailed herein. The treatment with the compound may be a component of a combination therapy or an adjunct therapy. The combination therapy can be sequential therapy, where the patient is treated first with one drug and then the other, or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier as are slow-release vehicles.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or topically onto a site of disease or lesion, all using dosage forms well known to those of ordinary skill in the pharmaceutical arts.

The compounds used in the method of the present invention can be administered in admixture with suitable pharmaceutical diluents, extenders, excipients, or in carriers such as the novel programmable sustained-release multi-compartmental nanospheres (collectively referred to herein as a pharmaceutically acceptable carrier) suitably selected with respect to the intended form of administration and as consistent with conventional pharmaceutical practices. The unit will be in a form suitable for oral, nasal, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms that can be useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as LNPs, small unilamellar vesicles, large unilamellar vesicles, and multilamellar vesicles. Liposomes can be formed from a variety of phospholipids such as lecithin, sphingomyelin, proteolipids, protein-encapsulated vesicles or from cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar-coated or film-coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials such as solutol and/or ethanol to make them compatible with the type of injection or delivery system chosen.

The compounds and compositions of the present invention can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by topical administration, injection or other methods, to the afflicted area, such as a wound, including ulcers of the skin, all using dosage forms well known to those of ordinary skill in the pharmaceutical arts.

Specific examples of pharmaceutically acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

The active ingredient can be administered orally in solid dosage forms, such as capsules, tablets, powders, and chewing gum; or in liquid dosage forms, such as elixirs, syrups, and suspensions, including, but not limited to, mouthwash and toothpaste. It can also be administered parentally, in sterile liquid dosage forms.

Solid dosage forms, such as capsules and tablets, may be enteric-coated to prevent release of the active ingredient compounds before they reach the small intestine. Materials that may be used as enteric coatings include, but are not limited to, sugars, fatty acids, proteinaceous substances such as gelatin, waxes, shellac, cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), and methyl methacrylate-methacrylic acid copolymers.

The compounds and compositions of the invention can be coated onto a solid support for temporary or permanent implantation into the bone marrow of a subject.

As used herein, to treat myelofibrosis in a subject who has myelofibrosis means to stabilize, reduce, ameliorate or eliminate a sign or symptom of myelofibrosis in the subject. Symptoms of myelofibrosis include tiredness and shortness of breath—due to low numbers of red blood cells; bleeding and bruising easily—due to low numbers of platelets; pain and discomfort in the stomach (abdomen) due to enlarged spleen and liver; bone pain; gout/painful, stiff or swollen joints; loss of appetite and weight loss; fever; night sweats; very itchy skin (pruritus). The myelofibrosis can be a primary myelofibrosis or can be a secondary myelofibrosis.

All combinations of the various elements described herein are within the scope of the invention unless otherwise indicated herein or otherwise clearly contradicted by context.

Where a numerical range is provided herein, it is understood that all numerical subsets of that range, and all the individual integers contained therein, are provided as part of the invention. Thus, for example, a dose which is from 10-100 mg includes the subset of doses which are 10 to, the subset of doses which are 20 to 75 mg etc. as well as a dose which is 51 mg, a does which is 78 mg, a dose which is 90 mg, etc. up to and including a dose which is 100 mg.

### Results

Here we systematically analyzed the bone marrow stromal compartment using genetic lineage tracing and single-cell RNA sequencing (scRNA-seq) in two different PMF models (TOE and MPLW515L). We found that LepR-lineage cells gave rise to the majority of collagen-expressing myofibroblasts throughout the bone marrow. In contrast, Gli1-lineage cells represented a minority of stromal cells that underwent myofibroblastic differentiation and were largely confined to areas of active osteoblastogenesis. Gli1 deletion, from either the hematopoietic or stromal compartment, did not affect PMF pathogenesis, including bone marrow fibrosis. Consistently, unbiased scRNA-seq demonstrated that Lepr+ MSCs were the major perpetrators of bone marrow fibrosis. In PMF, LepR+ cells expanded, lost their HSC-supporting capacity and transformed into factories for fibrosis and extracellular matrix (ECM) remodeling proteins. This mesenchymal remodeling was associated with a sinusoidal-to-arteriolar conversion of the vasculature. We also identified expanded populations of pericytes and Sox10+ glial cells, which appeared to serve as a neurovascular signaling hub. Ablation of bone marrow glial cells significantly reduced bone marrow fibrosis and other pathology in PMF. Our work firmly establishes the fibrogenic role of LepR+ MSCs and identifies glial cells as a therapeutic target in the PMF bone marrow.

LepR-Lineage Cells are Distributed Throughout the Bone Marrow while Gli1-Lineage Cells Reside Primarily Near the Growth Plate at Steady State:

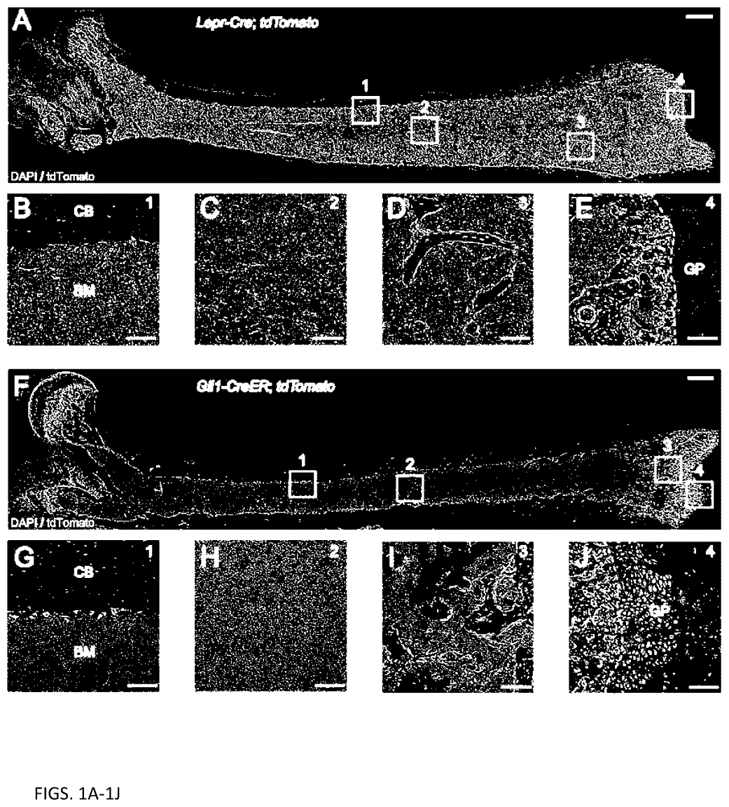

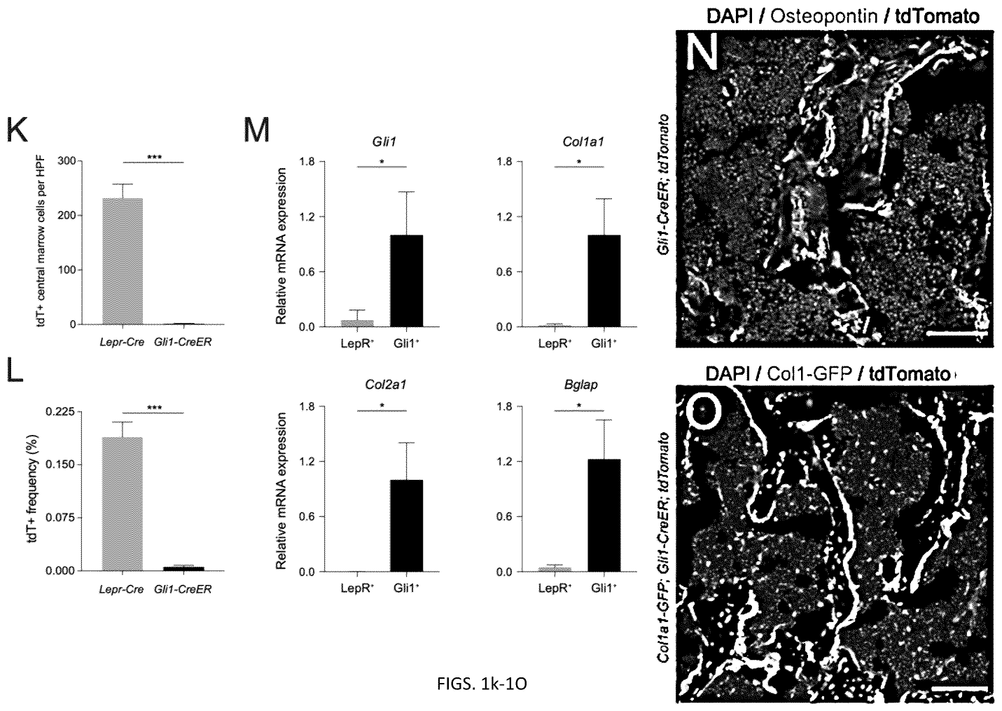

We performed lineage tracing of long bones from Lepr-Cre; loxp-tdTomato and Gli1-CreER; loxp-tdTomato mice to delineate the localization of these two MSC populations at steady state. In young adult Lepr-Cre; loxp-tdTomato mice, tdTomato+ cells were distributed uniformly throughout the marrow cavity in the expected perivascular arrangement with elongated cellular processes consistent with their MSC identity (FIGS. 1A-1C). There were also tdTomato+ cells below the growth plate and lining nascent trabecular bone in the metaphysis (FIGS. 1D and 1E). Concurrent examination of Gli1-CreER; loxp-tdTomato mice was carried out using an induction strategy at 6-7 weeks of age as described previously 19. Three doses of tamoxifen were administered by oral gavage on alternating days followed by analysis after varying chase periods (48 hours, 1 month, and 1 year). Assessment at 48 hours, when labeling should be largely restricted to cells with active Hedgehog (Hh) signaling, revealed several distinct domains of labeling: articular cartilage, superior growth plate chondrocytes, growth plate perichondrium, and primary spongiosa as well as sparse cells along the endocortical interface and surrounding larger blood vessels. At 1 month after Cre induction, these domains were preserved with some expansion below the growth plate and extension into the metaphysis (FIGS. 1F-1J). Nascent trabecular bone was lined by numerous tdTomato+ cells (FIG. 1I). Columns of chondrocytes spanning the growth plate were also tdTomato+ (FIG. 1J). In stark contrast to LepR labeling, perisinusoidal MSCs in the central marrow were not labeled in Gli1-CreER; loxp-tdTomato mice (FIG. 1H). Quantification of tdTomato+ cells by microscopy and flow cytometry confirmed the disparity in labeling of central marrow MSCs between Lepr-Cre and Gli1-CreER (FIGS. 1K and 1L). Compared with LepR-lineage cells, Gli1-lineage cells appeared to adopt more of an osteolineage fate based not only on localization but higher expression of osteolineage genes such as Col1a1, Col2a1, and Bglap (FIG. 1M) and expression of osteopontin and Col1a1-GFP protein (FIGS. 1N and 1O). These observations reflect the established roles of Hh signaling in osteoblast differentiation and growth plate chondrocyte maintenance 20-23.

We further ensured that tdTomato labeling was specific to MSCs by crossing a Pdgfra-GFP reporter allele 24 with each of the above genotypes. LepR-lineage cells were virtually synonymous with Pdgfra-expressing cells in the bone marrow (>94% overlap). In contrast, at 48 hours after Cre induction, very few Pdgfra-GFP+ cells expressed tdTomato in the Gli1-CreER; loxp-tdTomato mice (<2%); however, the rare tdTomato+ cells from the marrow cavity were Pdgfra-GFP+, indicating their MSC origin. Similar labeling patterns were observed at 1 month and even 1 year after tamoxifen induction. Thus, LepR-lineage cells represent MSCs throughout the bone marrow while Gli1-lineage cells represent primarily osteolineage-committed MSCs concentrated near the growth plate at steady state.

### LepR- but not Gli1-Lineage Cells are the Predominant Source of Myofibroblasts in the TOE Model of PMF:

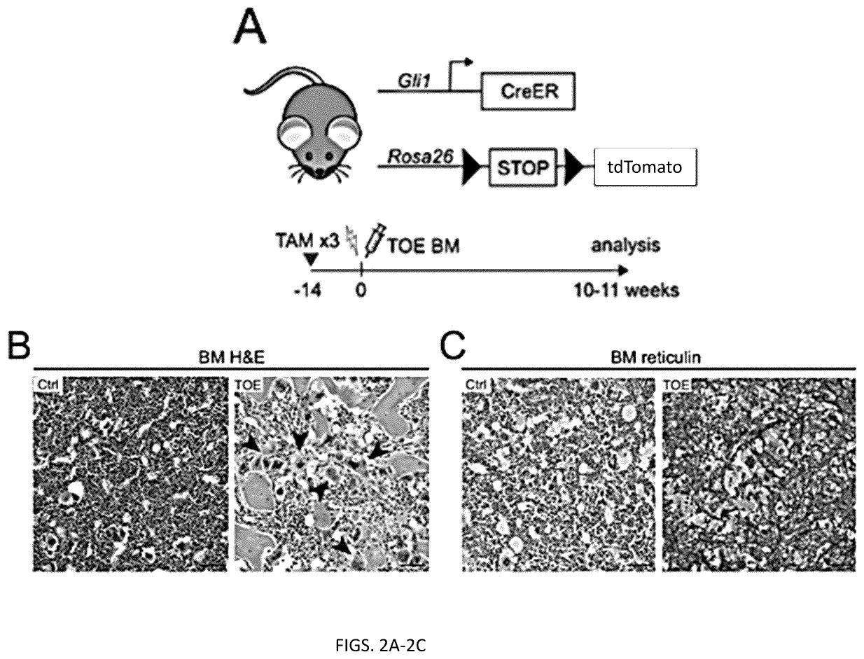

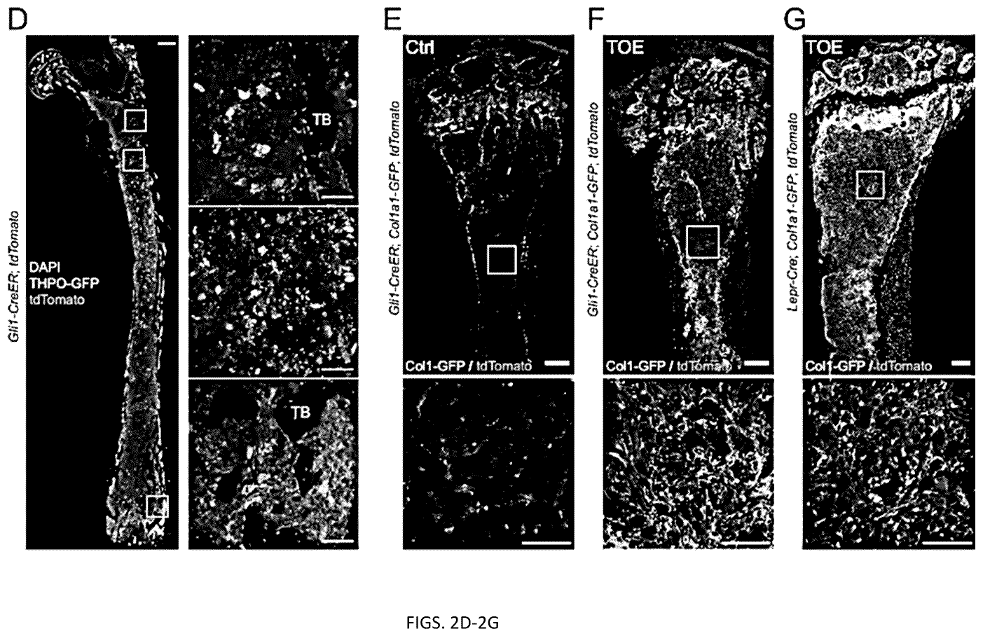

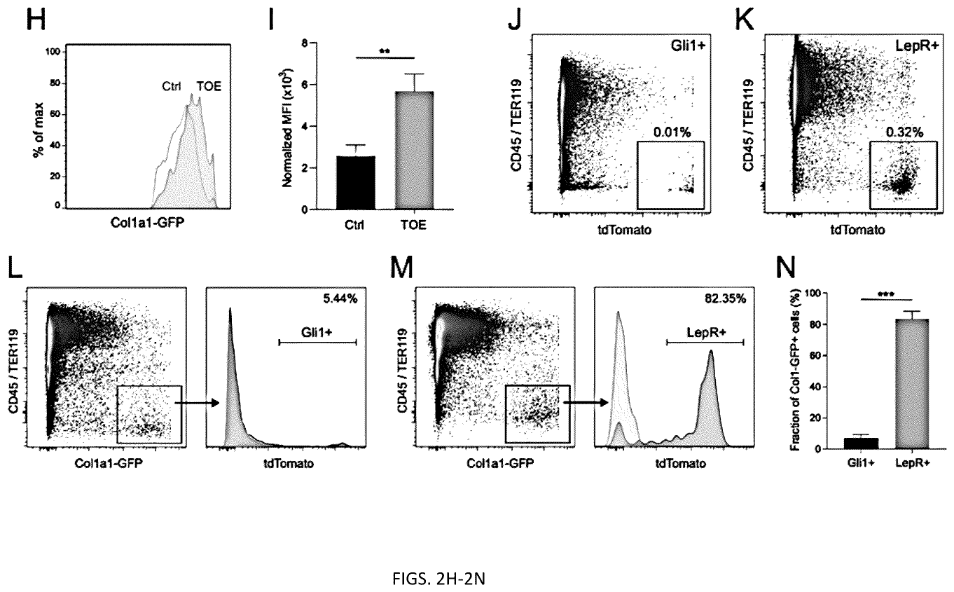

LepR+ and Gli1+ cells are putative MSCs giving rise to myofibroblasts in PMF 18,19, but the extent to which these cells contribute to myofibroblasts has yet to be directly compared. LepR- and Gli1-lineage cells occupy largely distinct areas of long bones aside from limited overlap in the metaphysis at steady state (FIG. 1). Knowing that LepR-lineage cells represent most MSCs in the central marrow and outnumber Gli1-lineage cells by over an order of magnitude, we reasoned that myofibroblasts derive predominantly from LepR+ MSCs. We used the TOE model to induce PMF because it has been previously used to examine MSC behavior in PMF and serves as a suitable comparator across studies 18,19. Gli1-CreER; loxp-tdTomato recipient mice were induced with tamoxifen 10-14 days before bone marrow transplantation (FIG. 2A). Wild-type donor bone marrow cells were retrovirally transduced with either TOE or empty vector to generate PMF and control mice, respectively. As expected, TOE mice displayed features of PMF such as increased proliferation of atypical megakaryocytes, osteosclerosis, and dense reticulin fibrosis (FIGS. 2B and 2C). To fully capture the changing PMF bone marrow stromal architecture, we performed confocal imaging on whole bone sections. Gli1-lineage cells expanded focally near areas of baseline localization (metaphyseal trabecular bone, endosteum) but remained largely absent from the central marrow in TOE mice (FIG. 2D). We then incorporated a Col1a1(HS4,5-3.2 kb)-GFP (Col1a1-GFP) reporter as a readout of collagen-producing myofibroblasts 25. Again, Gli1-lineage cells remained near the growth plate, without an evident presence in the central marrow where Col1a1-GFP+ myofibroblasts were abundant (FIGS. 2E and 2F). In stark contrast, a high degree of overlap was observed in Lepr-Cre; loxp-tdTomato; Col1a1-GFP recipient mice (FIG. 2G). Consistent with a myofibroblast identity, stromal cells significantly upregulated Col1a1-GFP expression throughout the marrow cavity (FIGS. 2H and 2I). In line with these data, flow cytometry showed that the frequency of tdTomato+ cells in Lepr-Cre; loxp-tdTomato mice significantly outnumbered those from Gli1-CreER; loxp-tdTomato mice in PMF (FIGS. 2J and 2K). Only about 5% of Col1a1-GFP+ myofibroblasts from Gli1-CreER; loxp-tdTomato; Col1a1-GFP recipient mice expressed tdTomato. Over 80% of Col1a1-GFP+ cells expressed tdTomato in Lepr-Cre; loxp-tdTomato; Col1a1-GFP mice (FIGS. 2L-2N), indicating that LepR-lineage cells comprise the majority of bone marrow myofibroblasts.

Non-inducible Lepr-Cre permanently labels all LepR+ cells and their progeny from embryogenesis onwards. This absence of temporal control can confound the interpretation of lineage tracing results, although previous studies have demonstrated that LepR expression persists in adult bone marrow MSCs 11,16. We generated Lepr-CreER knock-in mice in an attempt to overcome this limitation. Tamoxifen induction specifically labeled bone marrow stromal cells in Lepr-CreER; loxp-tdTomato mice, albeit with limited efficiency. Consistent with our Lepr-Cre results, an expansion of adult LepR+ cells was also observed in the PMF bone marrow.

To synchronously delineate the contribution of Gli1-lineage cells to myofibroblasts in PMF, we performed dual linage tracing using a Pdgfra-DreER knock-in allele 26. Tamoxifen treatment induced specific labeling of bone marrow stromal cells in Pdgfra-DreER; rox-GFP mice. We then generated Gli1-CreER; loxp-tdTomato; Pdgfra-DreER; rox-GFP mice and induced PMF by transplanting TOE bone marrow cells. Although expansion of Pdgfra-GFP+ MSCs was readily detected in the bone marrow, these cells were tdTomato−, suggesting that Gli1-lineage cells are not a major source of Pdgfra-lineage myofibroblasts in PMF.

### Stromal Gli1 is Dispensable For Bone Marrow Fibrosis And PMF Progression:

Besides its utility as an MSC marker, Gli1 and the associated Hh signaling pathway have garnered interest as therapeutic targets in PMF. Early investigation focused on Hh signaling in hematopoietic cells, but more recently, there has been growing interest in the role of Hh signaling in the stroma 19. Pharmacologic inhibition of Gli proteins has been observed to ameliorate fibrosis in a number of fibrosis models, including the JAK2V617F PMF model 19. Given that Gli1+ cells contribute to only a minority of bone marrow myofibroblasts, we wondered whether occult Gli1 activity (i.e., not detected using Gli1-CreER) or perhaps disease context-specific Gli1 activation could explain the disproportionate therapeutic impact. To address these questions, we obtained Gli1 knockout mice and backcrossed them onto a C57BL/6 background 27. Gli1 was efficiently deleted and no overt change in Gli2 expression was observed in Gli1−/− mice. These mice exhibited normal blood cell counts, bone marrow cellularity, and spleen weight compared with controls. In addition, the frequencies of Lin-Sca1+ cKit+ (LSK) hematopoietic progenitors, LSKCD150-CD48− multipotent progenitors (MPPs), and LSKCD150+CD48− HSCs were comparable to Gli1+/− and wild-type controls. Therefore, Gli1 is not required for steady-state adult hematopoiesis, a finding that aligns with prior studies 28,29.

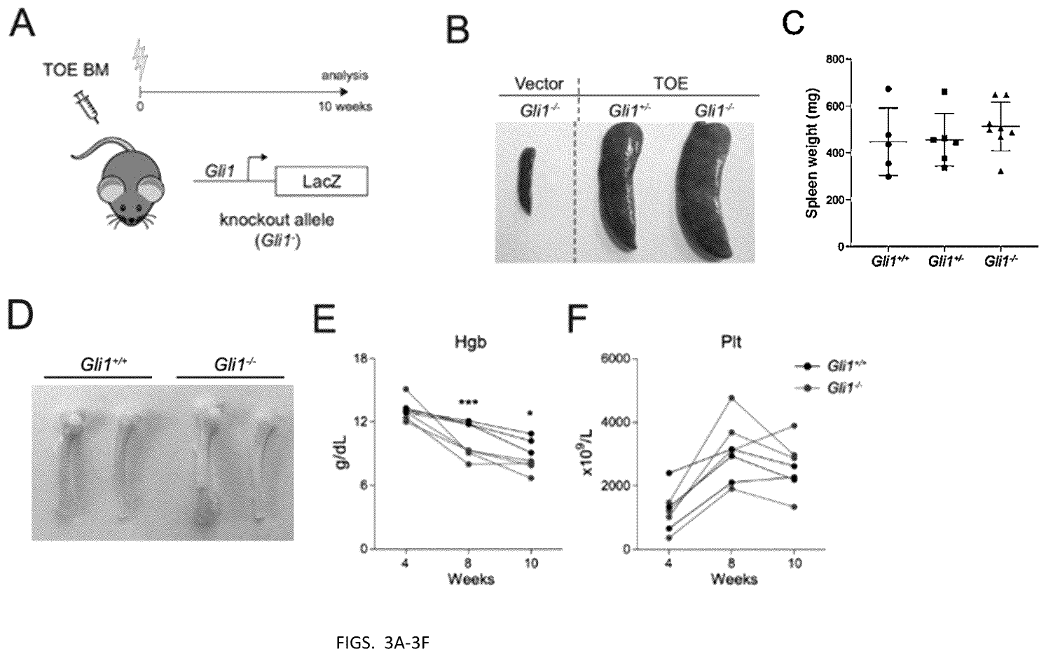

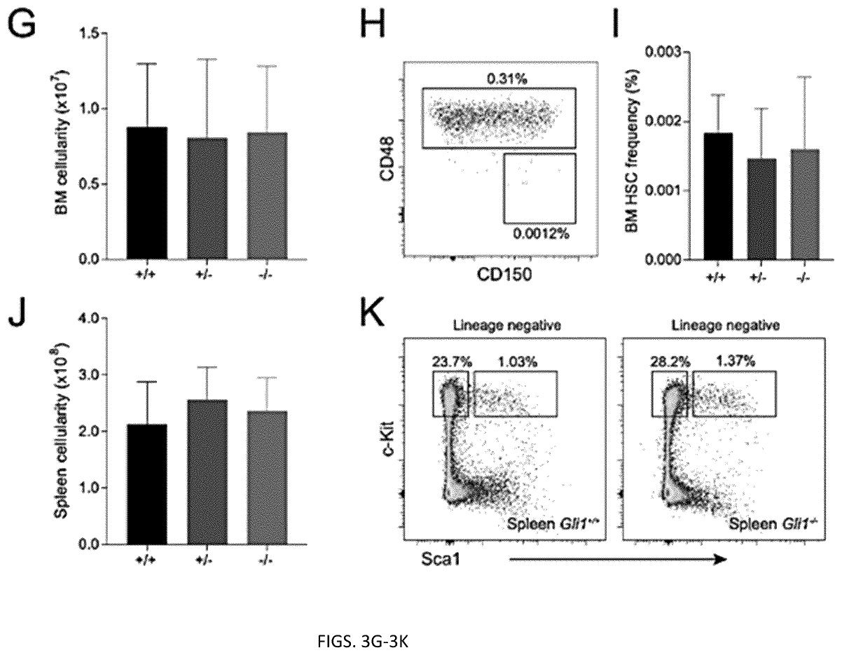

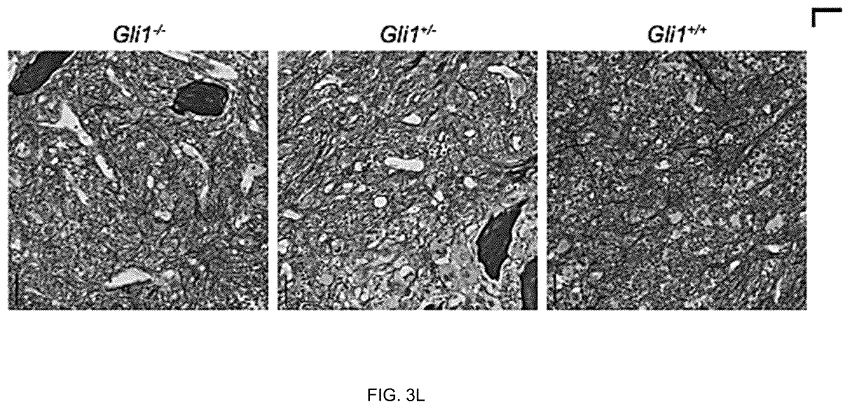

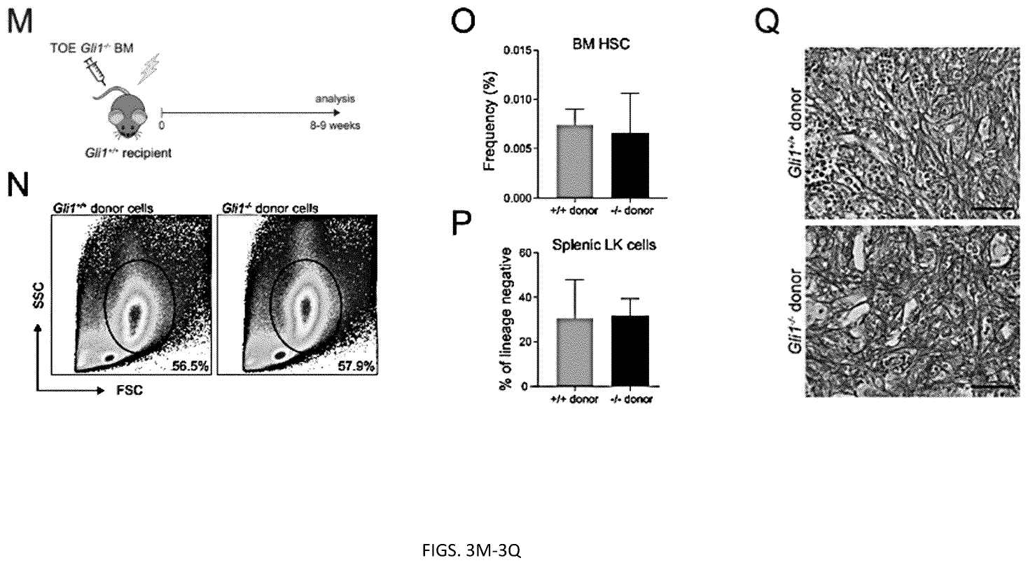

We next assessed stromal Gli1-dependence in PMF by transplanting TOE bone marrow cells into lethally irradiated Gli1−/− and control mice (FIG. 3A). Mice with stromal Gli1 deletion had similar splenomegaly, bone marrow abnormalities, and platelet counts compared with controls, although hemoglobin levels were modestly reduced (FIGS. 3B-3F). These mice also exhibited equivalent reductions in bone marrow cellularity and HSC frequency and increases in spleen cellularity and hematopoietic progenitor frequency (FIGS. 3G-3K). Importantly, mice with a Gli1−/− stromal compartment developed bone marrow fibrosis to a similar extent as controls (FIG. 3L). These data suggest that microenvironmental Gli1 is dispensable for PMF pathology.

### Hematopoietic Gli1 is not Required for PMF Pathogenesis:

It has also been proposed that aberrant Hh signaling in malignant hematopoietic cells may contribute to PMF pathology 19,30. As a direct transcriptional mediator, Gli1 is an indicator of Hh activity. To evaluate whether Gli1 is expressed by hematopoietic cells in PMF, we transplanted TOE-transduced Gli1-CreER; loxp-tdTomato donor cells into Col1a1-GFP recipient mice and administered tamoxifen shortly before analyses. Despite robust induction of myelofibrosis, no discernable tdTomato+ hematopoietic cells were observed in these mice. Similar results were observed with the MPLW515L PMF model. Thus, Gli1 and the Hh pathway are not activated in malignant hematopoietic cells to any significant degree in murine PMF.

We tested the contribution of hematopoietic Gli1 to PMF by transplanting TOE Gli1−/− bone marrow cells into lethally irradiated wild-type recipient mice (FIG. 3M). Mice with hematopoietic compartments reconstituted by Gli1−/− cells had similar blood cell counts, chimera levels, spleen sizes, and bone marrow and spleen cellularity compared with controls. These mice also had similar myeloproliferation and hematopoietic stem/progenitor cell frequencies relative to controls (FIGS. 30 and 3P). Bone marrow fibrosis was also similar in mice reconstituted with Gli1−/− bone marrow cells (FIG. 3Q). These data suggest that hematopoietic Gli1 is not required for the development of PMF pathology.

### LepR-Lineage Cells are the Major Source of Myofibroblasts in a Clinically Relevant MPLW515L PMF Model:

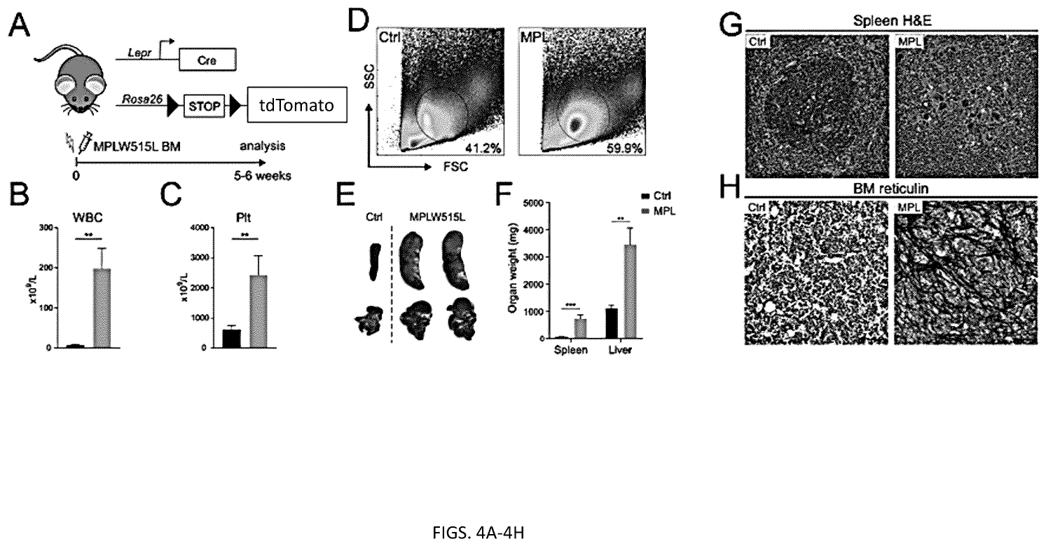

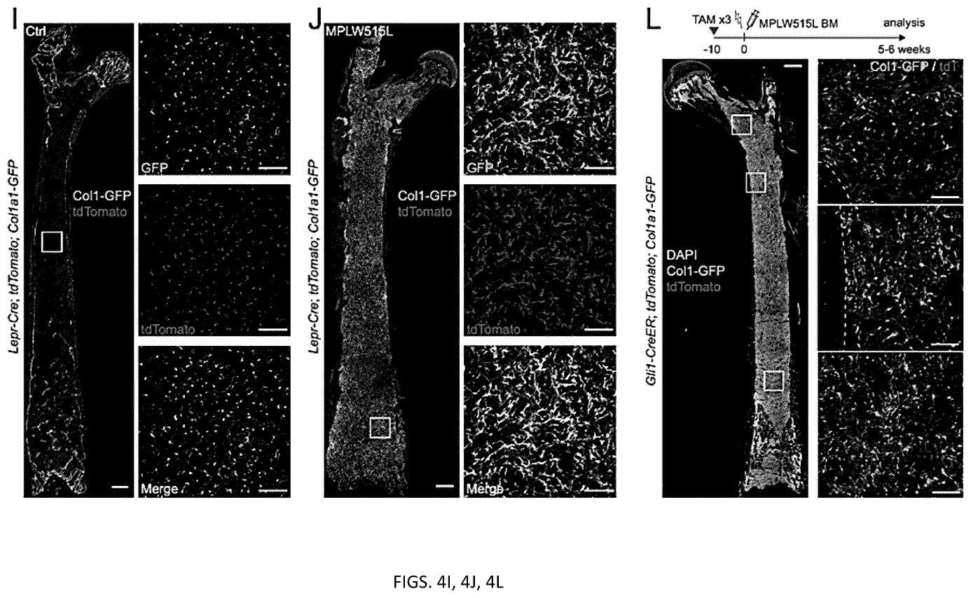

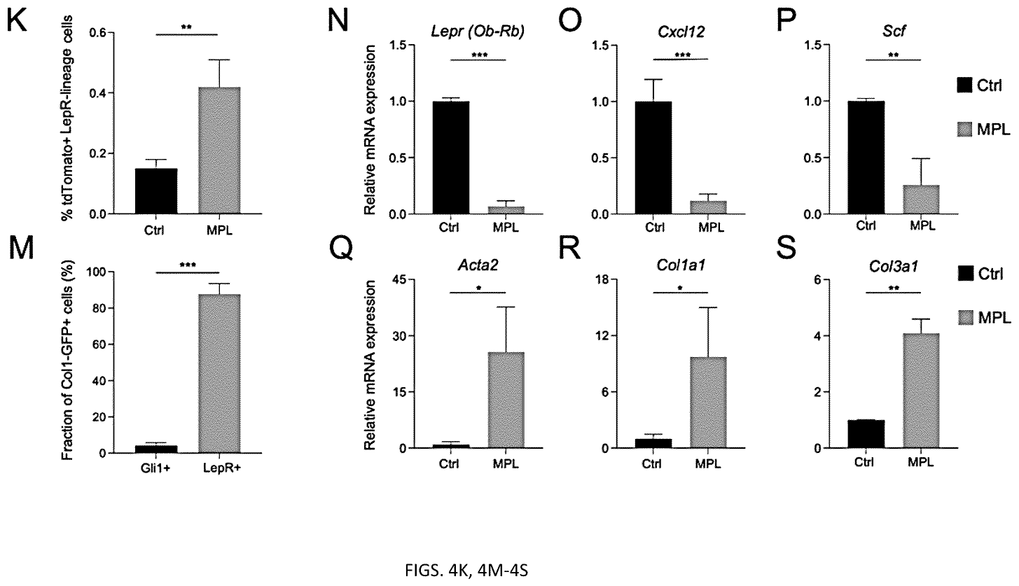

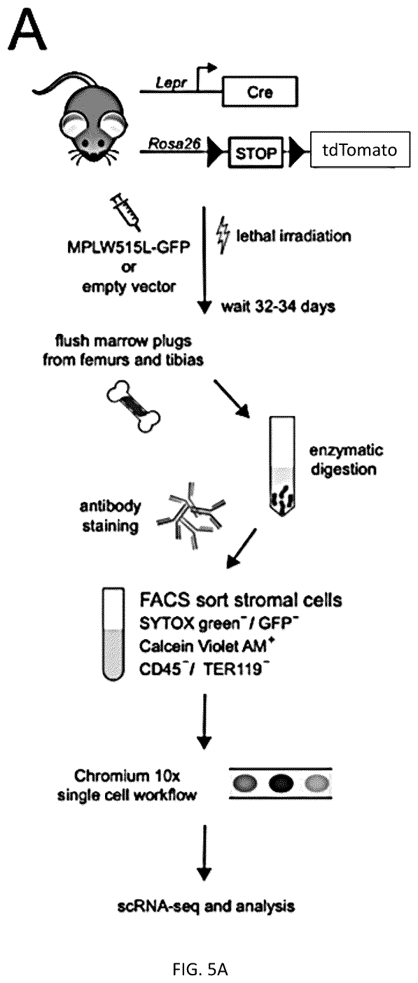

To validate our findings in a more clinically relevant model, we generated a PMF model using MPLW515L, a bona fide PMF driver mutation 1,3. Transplantation of MPLW515L-expressing bone marrow cells led to significantly increased white blood cell and platelet counts, and myeloproliferation at 5-6 weeks after transplantation (FIGS. 4A-4D). These mice also displayed hepatosplenomegaly with significantly increased spleen and liver weights (FIGS. 4E and 4F) and effacement of normal splenic architecture (FIG. 4G). Importantly, expression of MPLW515L led to prominent fibrosis in the bone marrow (FIG. 4H). Thus, MPLW515L mice developed robust features of PMF analogous to human patients, and consistent with earlier reports 3,31.

We then transplanted Lepr-Cre; loxp-tdTomato; Col1a1-GFP and Gli1-CreER; loxp-tdTomato; Col1a1-GFP mice with MPLW515L-transduced bone marrow cells and performed lineage tracing. Compared with controls, there was a significant expansion of LepR-lineage cells, which were predominantly Col1a1-GFP+ (FIGS. 4I-4K), suggesting that LepR-lineage cells are the major source of myofibroblasts. In contrast, Gli1-lineage cells were present near the growth plate with rare labeling along the endosteum of the diaphysis. No labeling was observed in most areas of the central marrow where Col1a1-GFP+ myofibroblasts were dramatically increased (FIG. 4L). Flow cytometry quantification showed that 88% of Col1a1-GFP+ stromal cells were from LepR-lineage cells while only 4% were from Gli1-lineage cells (FIG. 4M). Quantitative reverse-transcription PCR (qRT-PCR) analysis revealed that LepR-lineage cells downregulated Lepr as well as key HSC niche factors, Cxcl12 and Scf, in PMF (FIGS. 4N-4P). At the same time, these cells upregulated several fibrogenic genes, including Acta2, Col1a1, and Col3a1 (FIGS. 4Q-4S), suggesting that LepR-lineage cells transform from supporting HSCs to depositing excess ECM proteins in PMF. Altogether, our data demonstrate that LepR-lineage cell expansion and conversion to a myofibroblastic fate are shared features of PMF pathology in both TOE and MPLW515L models.

### Unbiased Single-Cell Transcriptional Profiling of Bone Marrow Stromal Cells Confirms Lepr-MSC Origin of Myofibroblasts:

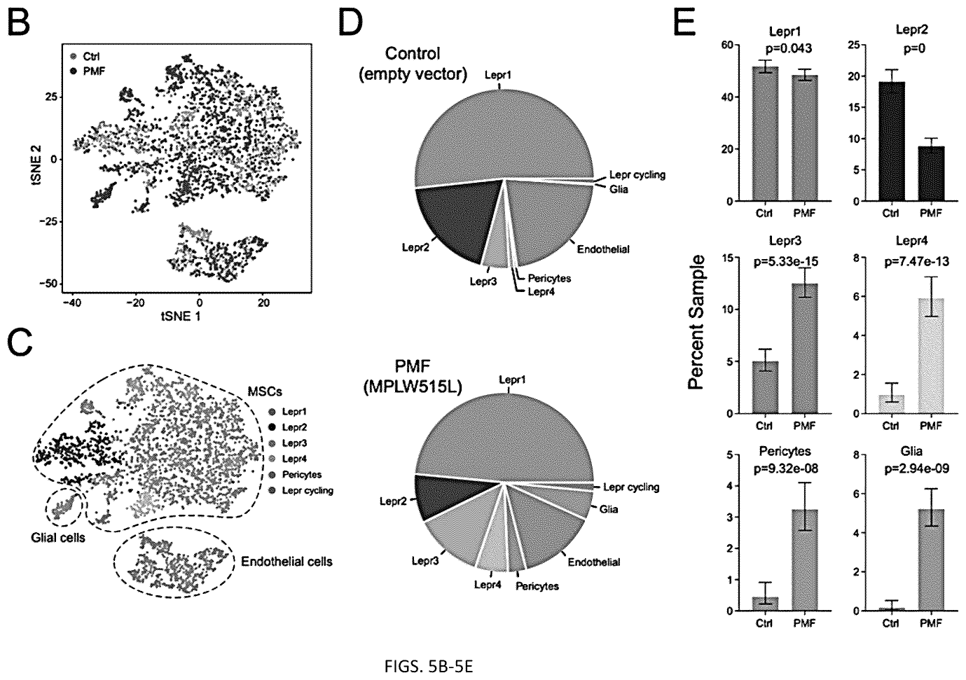

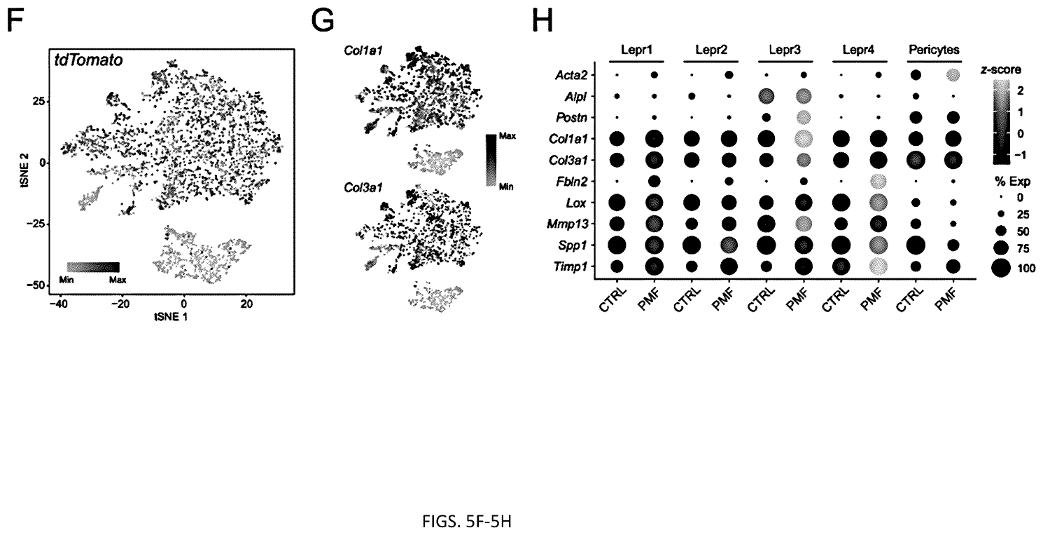

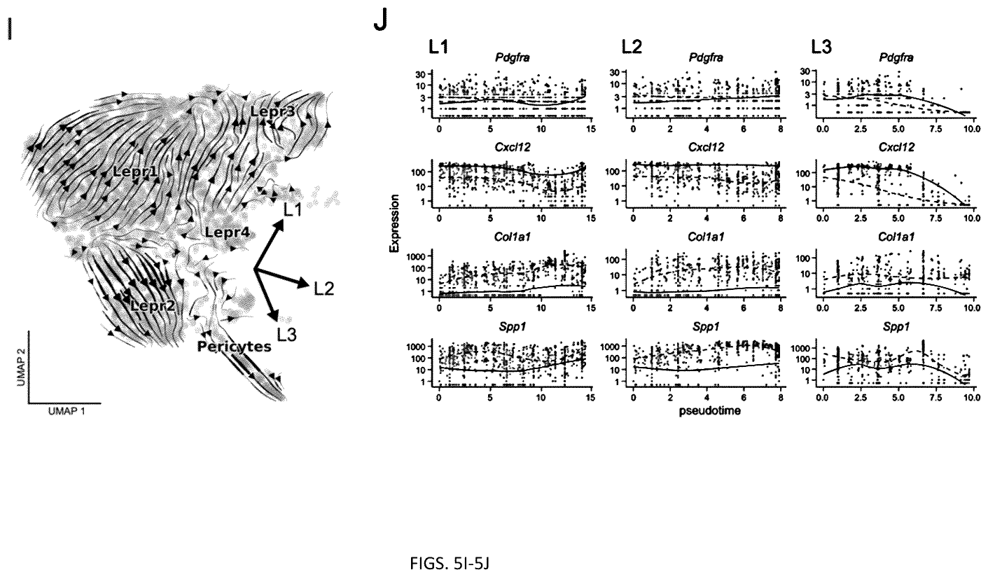

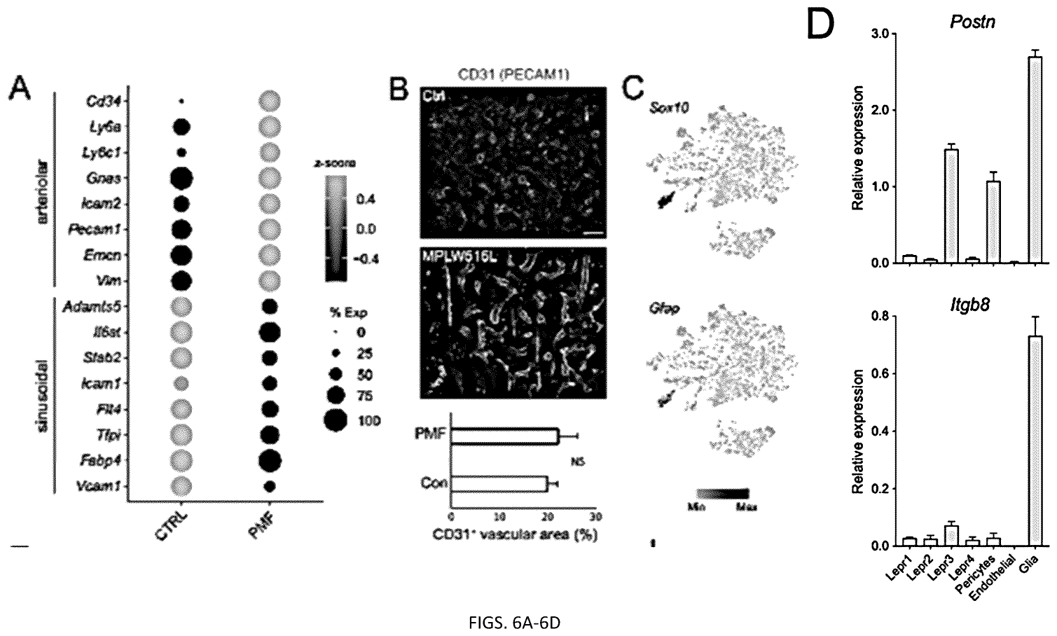

To comprehensively profile the complex stroma in PMF, we sorted CD45/Ter119—cells from the bone marrow of PMF and control Lepr-Cre; loxp-tdTomato mice (FIG. S5A) and performed scRNA-seq (FIG. 5A). Three distinct stromal cell lineages (MSCs, endothelial cells, and glial cells) were apparent from the aggregate data (FIGS. 5B and 5C). MSCs were the largest stromal population and could be further subdivided into Lepr1, Lepr2, Lepr3, Lepr4, Lepr cycling, and pericytes (FIG. 5C). Lepr1 was the most abundant and expressed the adipolineage gene Lp1, whose level was significantly downregulated in PMF (FIG. S5B). Lepr2 was distinguished by a prominent AP-1 pathway gene signature, with high expression of Fos, Jun, and Egr1 (FIG. S5B), suggesting an uncommitted state prior to differentiation 32. Lepr3 denoted osteolineage-fated cells based on higher expression levels of Alp1, Postn, Bglap, and Bglap2 (FIG. S5B). Lepr4 was enriched for matricellular genes, including Timp1, Fbln2, and Sdc4 (FIG. S5B), suggesting a prominent role in ECM remodeling. Compared with controls, PMF mice had significant expansion of Lepr3, Lepr4, pericytes, and glial cells accompanied by significant contraction of Lepr2 cells (FIGS. 5D and 5E). By including the tdTomato lineage marker in our experiments, we were able to demonstrate that Lepr-Cre is a pan-MSC marker in the bone marrow (FIG. 5F). Consistent with MSC transformation, Lepr+ cells downregulated expression of MSC markers and HSC niche factors (FIGS. S5B-S5D). No Gli1 could be reliably detected in the bone marrow stroma by scRNA-seq (FIG. S5C). Most LepR-lineage cells are perisinusoidal 7,33, but periarteriolar MSCs, commonly referred to as pericytes based on their specific expression of Meg3, Myh11, and Rgs5, were also derived from LepR-lineage cells (FIGS. 5F and S5B). These pericytes expressed lower amounts of Lepr and Pdgfra and were the highest expressors of Acta2 and Pdgfrb compared to other MSCs (FIGS. 5H and S5D). Thus, despite their phenotypic and functional differences, both perisinusoidal and periarteriolar MSCs are LepR-lineage cells. Importantly, across all stromal cells, LepR-MSCs were the major source of Col1a1, Col3a1, and other ECM proteins such as Mmp13, Spp1, Lox, and Timp1 (FIGS. 5F-5H). These data support our conclusion that LepR-lineage cells are the major precursors of myofibroblasts and drivers of bone marrow fibrosis in PMF.

We next focused on the expanded Lepr-MSC clusters to better define their differentiation trajectories in PMF. RNA velocity analysis revealed that Lepr+ MSCs underwent 3 primary lineage commitments in PMF: Li) osteolineage fate (Lepr3); L2) ECM remodeling fate (Lepr4); and L3) Acta2+ pericyte fate (FIG. 5I). Cell trajectory analysis identified similar lineage patterns. Gene ontology analysis also yielded biological process terms congruent with the cell fate commitments along these lineages. We plotted representative marker genes along pseudotime for each of the 3 lineages to discern temporal patterns. Although Pdgfra expression in PMF mirrored that in control MSCs, upregulation of ECM genes (e.g., Col1a1 and Spp1) was apparent at early stages and tended to increase over the continuum of cell states (FIGS. 5H and 5J). In a reciprocal fashion, HSC niche factors such as Cxcl12 were downregulated (FIG. 5J). Consistent with our lineage tracing and Gli1 knockout data (FIGS. 1-4), the Hh pathway was not significantly upregulated in any MSC populations. Overall, these data suggest that Lepr+ cells lose their MSC identity and adopt a myofibroblast identity to directly drive fibrogenesis in PMF.

Vascular endothelial cells skew toward an arteriolar gene expression signature in PMF

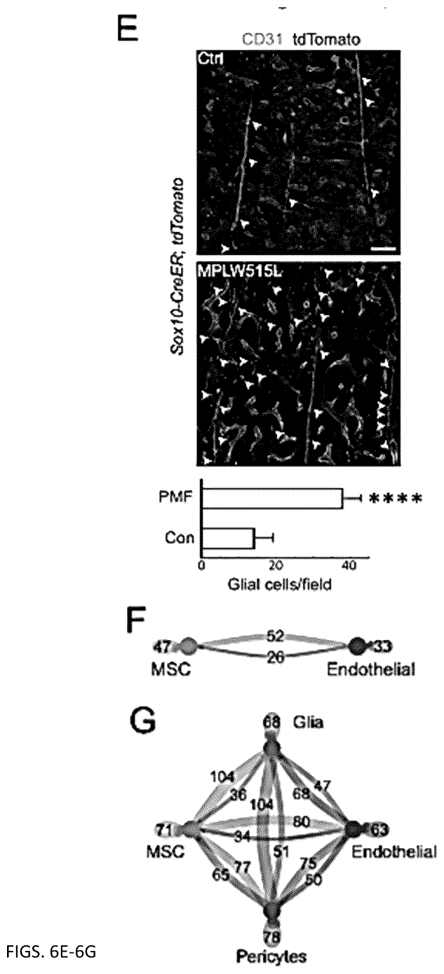

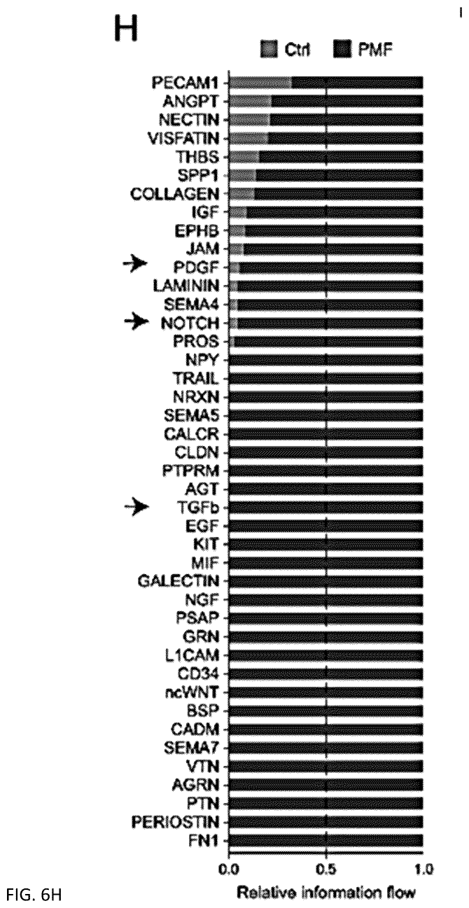

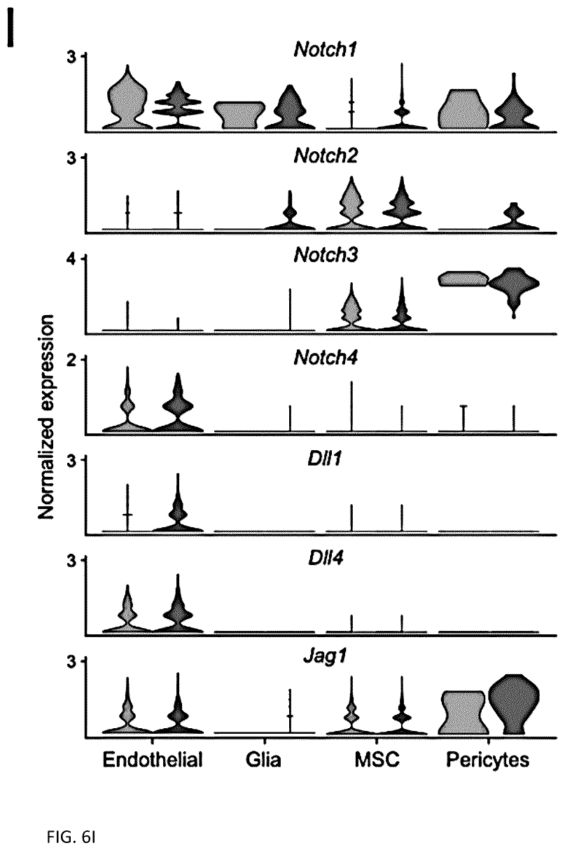

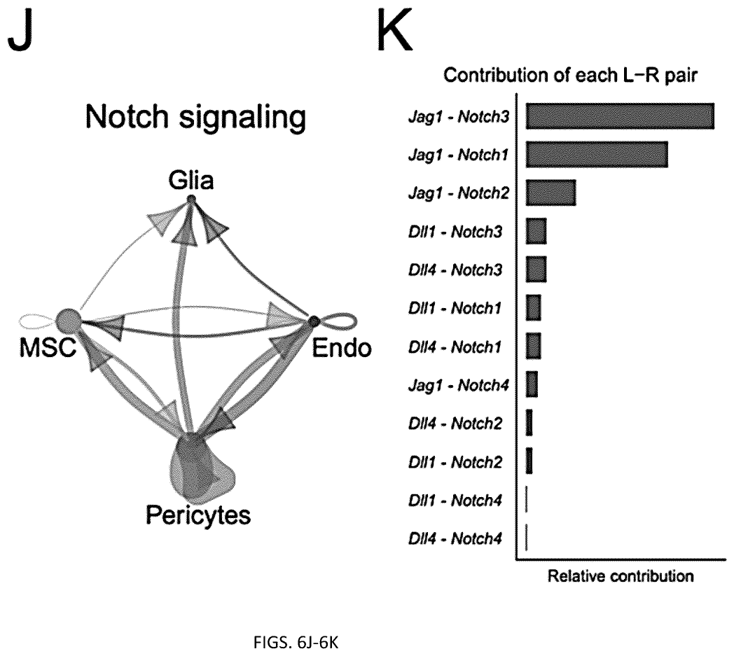

Endothelial cells are an important stromal cell type in the bone marrow, but how they behave in PMF remains poorly understood. Our single-cell analysis included endothelial cells (FIG. 5C). At steady state, the vasculature within the central marrow cavity consists primarily of a dense network of sinusoids that forms a key component of the perivascular HSC niche 34,35. These endothelial cells express lower levels of PECAM1 (also known as CD31) and endomucin (EMCN) and have been termed type L endothelial cells 36. Consistent with a sinusoidal identity, unperturbed bone marrow endothelial cells express sinusoidal-signature genes such as Flt4, Il6st, Stab2, and Vcam1 15-17. These markers were preferentially expressed by endothelial cells from control animals, indicative of normal sinusoidal regeneration and niche function after transplantation (FIG. 6A). On the contrary, endothelial cells from PMF mice downregulated these sinusoidal markers and expressed significantly higher levels of arteriole-associated genes such as Cd34, Pecam1, Ly6a, and Emcn (FIG. 6A), suggesting an endothelial fate change. Immunostaining confirmed that endothelial cells in the PMF bone marrow expressed higher levels of PECAM1 with more longitudinal branching (FIG. 6B), consistent with active neoangiogenesis and the resulting increase in bone marrow vascularity in PMF patients 37-39, although the vascular area was not significantly changed. Interestingly, bone marrow PECAM1high EMCNhigh type H endothelial cells have been reported to support osteoprogenitor differentiation and bone formation 36,40. It is thus conceivable that an arteriolar/type H fate change by the sinusoidal endothelial cells contributes to bone remodeling and the development of osteosclerosis in PMF.

scRNA-Seq Analysis Identifies an Expanded Sox10+ Glial Cell Population in PMF:

Our scRNA-seq analysis also identified a pronounced glial cell population in PMF (FIG. 5C). These cells specifically expressed markers associated with a glial identity, such as Sox10, Gfap, Apod, Plp1, and Ngfr (FIG. 6C). These cells were rare in normal bone marrow but expanded dramatically in PMF (FIG. 5E). The Sox10+ glial cells were Lepr− and tdTomato− (FIG. 5F), indicating a separate lineage origin from LepR-MSCs. Interestingly, Sox10+ glial cells expressed higher levels of periostin (Postn) than most Lepr+ MSCs (FIG. 6D). It has been reported that glial cells proliferate and migrate in response to Postn 41,42. Thus, the expansion of Sox10+ glial cells in PMF suggests a drastic remodeling of these cells driven by upregulated Postn in the bone marrow ECM, through a paracrine and/or autocrine manner. We also observed increased and specific expression of Erbb3 by Sox10+ cells, particularly in PMF (FIG. 6D). Since Erbb3 is a key gene mediating glial cell development, migration, and myelination 43-45, these data suggest that Sox10+ glial cells utilize the Erbb3 pathway to promote their expansion and remodeling in PMF. Steady state GFAP+ nonmyelinating Schwann cells promote HSC maintenance in the bone marrow by serving as a TGF-β activation center 13. Sox10+ cells also specifically expressed Itgb8 (FIG. 6D), an integrin that has been implicated in directly activating the latent form of TGF-β in the bone marrow 13, suggesting a possible model where availability of active TGF-β is regulated by these cells in PMF.

To further characterize these Sox10+ cells in vivo, we obtained a Sox10-CreER transgenic allele 46 and generated Sox10-CreER; loxp-tdTomato mice. Consistent with the periarteriolar localization of GFAP+ nonmyelinating Schwann cells 13, tdTomato+ cells were concentrated around bone marrow arterioles. Interestingly, these cells changed from a typical continuous branchless arrangement in the control marrow into a branching distribution in the PMF marrow (FIG. 6E). Consistent with our scRNA-seq data, quantification of bone marrow section images also revealed a significant increase of Sox10+ cells in PMF compared with controls (FIG. 6E). Given the central role of TGF-β as a stimulus for bone marrow fibrosis 47, these findings suggest that expanded Sox10+ glial cells may play an important role in promoting PMF pathology.

### Coordinated Remodeling of the Bone Marrow Stromal Compartment in PMF:

Given the close proximity of stromal cells, complex signaling likely coordinates the stromal remodeling in PMF. To gain insights into the coordinated cell fate changes in the PMF stroma, we performed ligand-receptor interaction analysis using CellChat 48. In control mice, modest cell-cell communication was evident and predominantly between MSCs and endothelial cells (FIG. 6F). Strikingly, in PMF, a dramatic increase in cell-cell signaling emerged amongst MSCs, endothelial cells, pericytes, and glial cells (FIG. 6G). Pericytes and glial cells appeared to be central signaling hubs within the bone marrow stroma (FIG. 6G).

Endothelial-specific activation of Notch in bone increases type H endothelial cell number, which in turn promotes osteogenesis 36,40. The Notch pathway also plays an important role in maintaining osteolineage cells 49 and osteoblast-specific activation of Notch leads to osteosclerosis 50. Thus, activated Notch promotes osteogenesis by acting on both endothelial cells and MSCs. We found that Notch signaling was significantly upregulated in PMF stromal cells compared with controls (FIG. 6H), suggesting a contribution by Notch to PMF. Similar to a previous report 16, endothelial cells served as the primary source of multiple Notch ligands, particularly Dll4, that acted via paracrine (on MSCs) and autocrine mechanisms in the control bone marrow (FIG. 6I). However, in PMF, pericytes significantly upregulated the Notch ligand Jag1 and exhibited more active Notch signaling (FIGS. 6I-6K), suggesting that pericytes may be the major effector cells relaying Notch-mediated bone remodeling in PMF.

Several signaling pathways with known roles in bone marrow fibrosis were also identified in PMF, including PDGF and TGF-β (FIG. 6H). The most active PDGF signaling was observed in Lepr-MSCs, suggesting a significant role of PDGF in directing MSCs toward a myofibroblast fate. This is consistent with the specific expression of Pdgfra/b by Lepr-MSCs. TGF-β signaling is a major mediator of bone marrow fibrosis and elevated levels of TGF-β have been frequently observed in both PMF animal models and patients 51,52. Deletion of Tgfb from hematopoietic cells abolishes bone marrow fibrosis in the TOE mouse model 47, suggesting that TGF-β is primarily elaborated by hematopoietic cells, in particular megakaryocytes 53. However, it is not clear what cells respond to TGF-β and how the pleiotropic effects of TGF-β signaling mediate bone marrow fibrosis in vivo. Interestingly, we found that within the bone marrow stroma, endothelial cells were the principal receivers of TGF-β signaling, suggesting that TGF-β may directly influence endothelial cells more than MSCs.

### Ablation of Bone Marrow Glial Cells Ameliorates PMF Pathology:

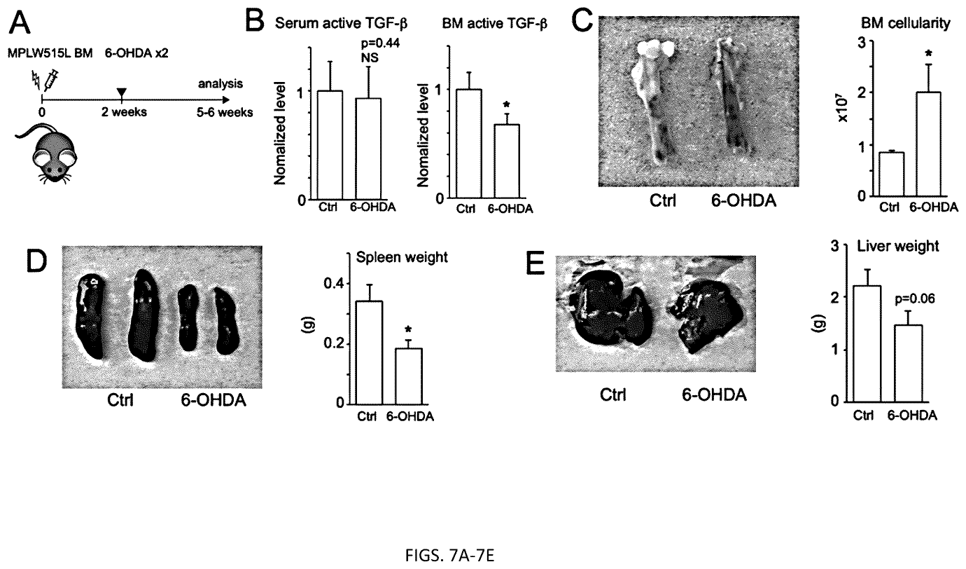

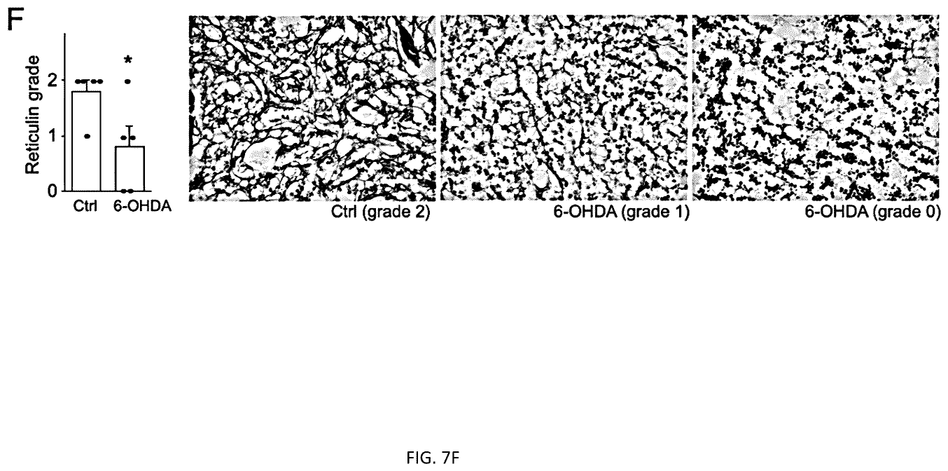

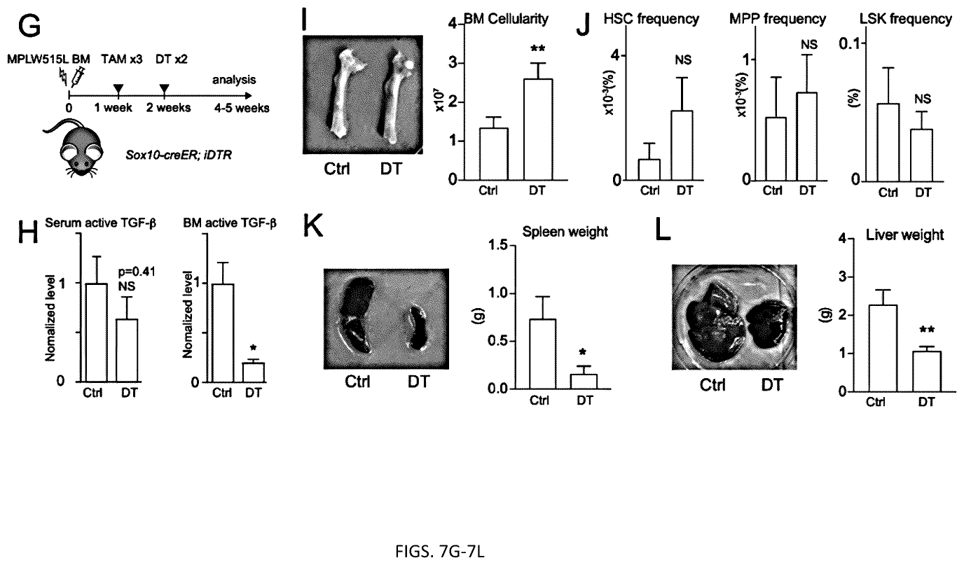

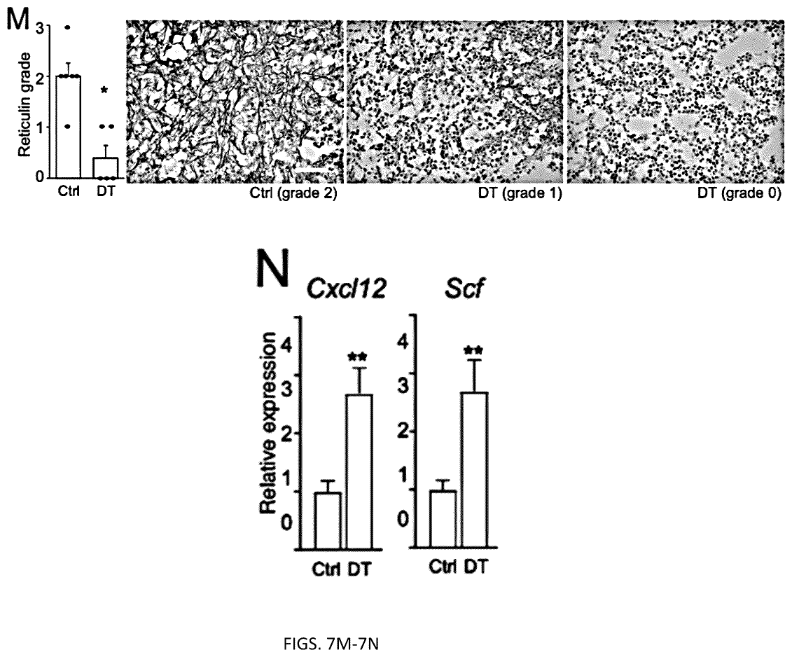

To assess the function of the expanded glial cells in PMF, we attempted to ablate these cells. Sympathectomy by transecting the sympathetic nerves leads to loss of bone marrow glial cells 13. To avoid potential complications associated with surgery, we performed chemical sympathectomy using 6-hydroxydopamine (6-OHDA) 54-57 (FIG. 7A). 6-OHDA-treated mice displayed no significant body weight loss with efficiently depleted Sox10+ glial cells in the bone marrow. Consistent with the notion that the glial cells may serve as the activation center for TGF-β in the bone marrow, ablation of these cells significantly reduced the levels of active TGF-β in the bone marrow without affecting the levels of active TGF-β in the serum (FIG. 7B). Ablation of bone marrow glial cells with 6-OHDA led to a significant increase in bone marrow cellularity in MPLW515L PMF mice (FIG. 7C). Treated MPLW515L PMF mice exhibited reduced spleen and liver sizes, suggesting a reduction in extramedullary hematopoiesis (FIGS. 7D and 7E). Importantly, bone marrow fibrosis in 6-OHDA-treated MPLW515L PMF mice was significantly reduced compared with vehicle-treated control MPLW515L PMF mice (FIG. 7F). Thus, chemical ablation of glial cells in the bone marrow ameliorates the pathology of PMF.

We also genetically ablated Sox10+ glial cells using Sox10-creER; iDTR (FIG. 7G). Administration of diphtheria toxin (DT) led to a significant body weight loss. In the bone marrow, DT effectively depleted Sox10+ glial cells but did not significantly impact HSC and progenitor frequencies in the bone marrow in steady state. However, active TGF-β in the bone marrow but not the serum was significantly reduced in DT-treated Sox10-creER; iDTR MPLW515L PMF mice (FIG. 7H). Ablation of Sox10+ glial cells led to a significant increase of bone marrow cellularity in treated Sox10-creER; iDTR MPLW515L PMF mice without significantly affecting hematopoietic progenitor frequencies, although HSC frequency was trending higher (FIGS. 71 and 7J). The spleen and liver sizes were significantly reduced in the treated mice, indicating reduced extramedullary hematopoiesis (FIGS. 7K and 7L). Bone marrow fibrosis was also significantly ameliorated in DT-treated Sox10-creER; iDTR MPLW515L PMF mice (FIG. 7M). Ablation of Sox10+ glial cells led to upregulation of HSC niche factors (Cxcl12 and Scf), key MSC fate regulators (Foxc1 and Ebf3), and ECM degradation enzymes (Mmp13, Adamts9, and Ctsh) in bone marrow MSCs (FIG. 7N), indicating restoration of HSC niche function and reversion of myofibrogenesis. Collectively, these data suggest that bone marrow glial cells play a critical functional role in promoting bone marrow fibrosis and targeting these cells may be a promising therapeutic strategy in PMF.

### DISCUSSION

How to reliably and reproducibly identify myofibroblasts remains a challenge. α-SMA (Acta2) is a commonly used marker 19, but it is not abundantly expressed by many ECM-producing myofibroblasts in the bone marrow (FIG. 5H). We propose that collagens, particularly Col1a1, may be more reliable and consistent markers of fibrosis (FIGS. 2, 4, and 5H), an observation supported by a recent comprehensive study of myofibroblasts in kidney fibrosis 58. Using a transgenic Col1a1-GFP reporter, we showed that LepR-lineage cells were the major source of myofibroblasts in PMF. In contrast, compatible with multiple previous reports 20-23,59, Gli1-lineage cells represented predominantly osteolineage cells at baseline and contributed to a minority of myofibroblasts in PMF (˜4-5%) (FIGS. 2 and 4). Since Gli1-lineage cells express PDGFRα and LepR-lineage cells are virtually synonymous with PDGFRα-expressing cells, Gli1-lineage cells are presumably a subset of LepR-lineage cells.

Treatment of PMF with Hh inhibitors has quickly advanced to clinical stage development despite conflicting data regarding the pathway's role in hematopoiesis 30. Because hematopoiesis occurs throughout the bone marrow 33 and Gli1-lineage cells constitute a minor portion of myofibroblasts near sites of active osteoblastogenesis (FIGS. 2 and 4), the local stromal changes contributed by Gli1-lineage cells are unlikely to play a prominent role in PMF. Furthermore, Hh signaling remained dormant in the context of PMF, and deletion of Gli1 from either the stromal or hematopoietic compartment did not impact PMF development (FIG. 3). These data undermine the value of Gli1+ cells and Gli1, the transcription factor, as therapeutic targets in PMF and shed light on why efforts to target Hh have yielded disappointing results in PMF 60-62.

Leimkuhler et al. also characterized PMF bone marrow stroma using scRNA-seq 63 and identified Lepr+ MSCs as the predominant fibrosis-driving cells. They isolated stromal cells by crushing the bone, which in our experience can introduce cells outside the bone marrow that are not directly relevant to myelofibrosis. In contrast, we confined our analysis to bone marrow stromal cells and characterized the stroma by incorporating the Lepr-Cre; tdTomato lineage marker and including endothelial cells. A notable difference in our study was the absence of the alarmin complex (S100A8/A9) in stromal cells. As ambient S100A8/A9 RNAs from monocytes are a significant source of contamination in droplet-based scRNA-seq 64, methods independent of droplet-based scRNA-seq are needed to confirm the stromal expression of alarmin genes in PMF. Furthermore, although inhibition of the alarmin complex using tasquinimod ameliorated PMF features 63, to what extent the effects were mediated through stromal cells remains unclear. One additional inconsistency was the attributed MSC origin for the Schwann cell precursors by Leimkuhler et al. 63. Using the tdTomato marker, we show that glial cell expansion occurred independently from LepR-lineage MSCs (FIG. 5F).

We show that Sox10+ glial cells are critical for PMF development and ablation of Sox10+ glial cells reduces the levels of activated bone marrow TGF-β (FIG. 7), indicating that TGF-β signaling is a mechanism by which these cells contribute to PMF. Although TGF-β signaling has been shown to be essential for bone marrow fibrosis, it does not significantly impact myeloproliferation in PMF, at least in the TOE model 47. Interestingly, inhibition of TGF-β signaling in MSCs blocks bone marrow fibrosis without affecting the expression HSC niche factors in PMF 65, suggesting that distinct molecular mechanisms control fibrosis and niche cell fate, and TGF-β signaling is only part of the PMF pathogenesis mechanism. Sox10+ glial cells are a signaling hub in the PMF stroma (FIG. 6G) and ablation of Sox10+ cells also increases the expression of HSC niche factors and ECM degradation enzymes in MSCs (FIG. 7), suggesting that Sox10+ cells promote PMF through multiple mechanisms.

### Experimental Model and Subject Details

### Mouse Strains

All mice were housed in specific pathogen-free, Association for the Assessment and Accreditation of Laboratory Animal Care (AAALAC)-approved facilities at the Columbia University Irving Medical Center. Food and water were provided ad libitum over 12-hour light/dark cycles. Lepr-Cre (JAX stock #032457)66, Gli1-CreER (JAX stock #007913)66, Gli1-LacZ (JAX stock #008211)27, Rosa26-loxp-stop-loxp-tdTomato (Ai9, JAX stock #007909)67, Rosa26-loxp-stop-loxp-iDTR (JAX stock #007900)68, Pdgfra-H2B-GFP (JAX stock #007669)24, and Sox10-CreER (JAX stock #027651)46 mice were obtained from the Jackson Laboratory. Col1a1(HS4,5-3.2 kb)-GFP25 and Pdgfra-DreER26 were described previously. Rosa26-rox-stop-rox-eGFP (RC::RG) mice were generated by crossing RC::RLTG mice (JAX stock #026931)69 with E2a-Cre mice (JAX stock #003724)69 to induce germline removal of the loxp-flanked tdTomato cassette from the Rosa26 locus. Young adult mice (both males and females of 6-12 weeks old) were used in this study. All experimental protocols were approved by Columbia University's Institutional Animal Care and Use Committee.

Generation of Lepr-CreER knock-in mice. The targeting vector for Lepr-CreER knock-in mice was generated and validated by sequencing (VectorBuilder). To achieve a similar recombination pattern as the Lepr-Cre mouse line, which is specific for the long isoform (Ob-Rb) of Lepr, IRES-CreER was inserted into the 3′ UTR of the endogenous Lepr locus analogous to 70. The targeting vector was electroporated into KV1 ES cells. Correctly targeted ES cells were identified through PCR analysis. Targeted ES cells were injected into blastomere to obtain chimera mice, which were bred to achieve germline transmission. Neo cassette was removed by mating with Flpe mice 71. After backcrossing, the mice were maintained on a C57BL/6J background.

### Method Details

Tamoxifen administration. Tamoxifen (Sigma T5648 or Cayman Chemical 13258) was dissolved in corn oil at a concentration of 40 mg/mL and stored at −20° C. until use. A total of 3 doses was administered (100 μl or 4 mg) once every other day by oral gavage. To induce Gli1-CreER, 6-7 weeks old mice were used as described in 19. Consecutive versus alternating day administration did not appear to significantly impact labeling efficiency. Additionally, both male and female mice were used for lineage tracing experiments and no sex-dependent differences were observed.

Retrovirus production and infection of bone marrow cells. The pMSCV-IRES-GFP (pMIG) backbone vector was used to generate pMIG-Thpo18 and pMIG-MPLW515L 3 plasmids as previously described. Ecotropic retroviral particles were produced via calcium phosphate-based cotransfection of HEK293T cells with the retroviral constructs and packaging plasmid (pCL-Eco, Addgene #12371). Viral supernatant was collected at 48 hours and 72 hours after transfection and subject to 0.22 μm filtration prior to spin infection of donor cells in the presence of 5 mg/ml polybrene (MilliporeSigma TR-1003-G). In preparation for donor cell transduction, fluorouracil (5-FU, 150 mg/kg) was administered to donor mice via intraperitoneal injection to enrich for stem and progenitor cells. Five days later, donor mice were euthanized and bone marrow cells were harvested from all hindlimbs. DMEM supplemented with 15% heat-inactivated fetal bovine serum (Sigma or Gemini), 100 ng/ml SCF, 10 ng/ml IL-3, 10 ng/ml IL-6 (PeproTech), 2 mM glutamine, 50 U/ml penicillin, 50 μg/ml streptomycin, and 50 μM 2-mercaptoethanol was used to maintain donor bone marrow cells in culture. Following overnight recovery, two spin infections on consecutive days were performed prior to transplant into lethally irradiated recipient mice (200 to 400 thousand cells per recipient). Transduction efficiency was consistently in the 40-60% range.

Bone marrow transplantation and induction of myelofibrosis. Young adult recipient mice were lethally irradiated using a Cesium 137 Irradiator (JL Shepherd and Associates) at 300 rad/minute with split doses of 525 rads (total 1050 rads) delivered at least 3 hours apart. Cells were transplanted by retro-orbital venous sinus injection of anesthetized recipient mice. Mice were maintained on antibiotic water (Baytril 0.17 g/l) for 14 days then switched to regular water. Recipient mice were periodically bled to assess the level of donor-derived blood cells (by flow cytometry).

Flow cytometry. Hematopoietic bone marrow cells were isolated by flushing hindlimb bones with HBSS (Ca²⁺ and Mg²⁺ free) with 2% heat-inactivated bovine serum (FACS buffer). Spleen cells were isolated by crushing the organ between the frosted ends of two glass slides. The cells were passed through a 25G needle several times and filtered with a 70 μm nylon mesh. The following antibodies were used to stain HSCs and HSPCs: anti-CD150 (TC15-12F12.2), anti-CD48 (HM48-1), anti-Sca-1 (E13-161.7), anti-cKit (2B8), anti-CD34 (RAM34), anti-CD16/32 (clone 93), and lineage markers (anti-Ter119, anti-B220 (6B2), anti-Gr1 (8C5), anti-CD2 (RM2-5), anti-CD3 (17A2), anti-CD5 (53-7.3), anti-CD8 (53-6.7)). Stromal cells were isolated from hindlimb bones by careful flushing to minimize tissue disruption using FACS buffer. Intact bone marrow plugs were digested with a mixture of Collagenase IV (200 U/ml, Worthington), Liberase DH (0.3 Wünsch units/ml, MilliporeSigma), and DNase I (200 U/ml, MilliporeSigma) diluted in HBSS (with Ca²⁺ and Mg²⁺) at 37° C. for 25 minutes. Samples were then dissociated by repetitive pipetting with a P1000 tip and filtered through a 40 μm cell strainer. MSC staining was performed for 30 minutes on ice with the following antibodies: anti-CD140a-biotin (APA5), anti-CD31 (MEC13.3), anti-CD45 (30F-11), and anti-Ter119. For flow cytometric analysis of peripheral blood, samples were subjected to ammonium chloride potassium red cell lysis before antibody staining. Antibodies including anti-Gr1 (8C5), anti-Mac-1 (M1/70), anti-B220 (6B2), and anti-CD3 (KT31.1) were then added to stain cells. FACS buffer with 1 μg/ml DAPI was added to samples shortly before analysis to exclude dead cells. All samples were run on BD FACSAria II or FACSCelesta flow cytometers. Data were analyzed using FlowJo (FlowJo LLC) software.

Bone sectioning and immunostaining. Freshly dissected long bones were fixed in 4% paraformaldehyde (PFA) overnight at 4° C. A shorter incubation period of 3-4 hours was used for antigens sensitive to extended fixation. Partial decalcification was then carried out in 10% EDTA at pH 7.4 for 3-4 days followed by overnight infiltration with 30% sucrose for cryoprotection. The bones were embedded in optimal cutting temperature (OCT) compound and stored at −80° C. Sections of 7 μm thickness were prepared using a CryoJane system (Instrumedics), dried overnight at room temperature, and stored at −80° C. until further use. Prior to immunostaining, sections were re-hydrated in PBS for 10 minutes and then blocked/permeabilized in a mixture of 0.3% Triton X-100 and 5% normal serum from the same species as the intended secondary antibody (goat or donkey) diluted in PBS for 1 hour. Primary antibodies were applied to the slides overnight at 4° C. followed by secondary antibody incubation for 1 hour at room temperature with repetitive PBS washes between steps. Slides were mounted with Prolong Gold Antifade (ThermoFisher) or VECTASHIELD (Vector Laboratories) and images were acquired on a Nikon Ti Eclipse scanning confocal microscope. Primary antibodies used were as follows: anti-Osteopontin (AF808, R&D, 1:100) and anti-CD31/PECAM1 (AF3628, R&D, 1:100).

Histology. Formalin-fixed, paraffin-embedded (FFPE) tissue specimens or frozen samples were prepared after fixation in 4% PFA. Sections of 4 or 7 μm were used for hematoxylin and eosin (H&E), reticulin and trichrome staining using standard methods. Fibrosis grading was performed according to a previously published scheme 72. Digital images were acquired using a Leica SCN400 brightfield microscope and processed using ImageScope viewer (Aperio).

6-hydroxydopamine treatment. PMF were induced by transplanting MPLW515L retrovirus infected bone marrow cells into recipient mice. Two weeks later, blood chimera were determined by flow cytometry. The mice were separated into two groups with similar chimeric levels. One group was treated with 6-OHDA (50 mg/kg in PBS with 0.01% ascorbic acid) while the other was treated with vehicle (0.01% ascorbic acid in PBS) twice in alternating days with intravenous injection. Two to three weeks later, the mice were analyzed.

Diphtheria toxin treatment. Diphtheria toxin dissolved in PBS was injected intraperitonially to treated mice at a dose of 500 ng/mouse in 100 ul on two consecutive days. Control mice were injected with PBS.

Active TGF-β ELISA. Blood was kept at room temperature for 30 min to 2 hs and then spun down twice at 2000 g for 5 min. Serum was collected and stored at −80° C. until analysis. Bone marrow fluid was collected by flushing one tibia and one femur with 200 ul FACS buffer using a 1 ml syringe with a 26G needle. The fluid was spun down at 1900 g for 5 min and the supernatant was store at −80° C. until analysis. ELISA was performed using Legend Max Free Active TGF-□1 ELISA kit from BioLegend (437707) following the instruction of the vendor.

Quantitative reverse transcription PCR. A minimum of 200 cells were FACS sorted directly into Trizol. Total RNA was extracted according to the manufacturer's instructions. All input material was used for first-strand cDNA synthesis (New England BioLabs ProtoScript II reverse transcriptase, M0368L). Quantitative real-time PCR was carried out using GoTaq qPCR Master Mix (Promega) on a Bio-Rad CFX Connect real-time PCR machine. β-actin was used to normalize DNA content across samples and relative gene expression was quantified using the 2-ΔΔCT method.

Single-cell preparation and sequencing. Bone marrow stromal cells were processed similarly to as described above with the addition of a live cell dye (Calcein Violet-AM, BioLegend) and dead cell dye (SYTOX Green, Thermo Fisher Scientific) after initial staining to exclude debris and ensure that only viable cells were sorted for downstream analysis. Immediately following FACS isolation, sample volume was reduced and single cells were encapsulated into emulsion droplets using Chromium Controller (10× Genomics). scRNA-seq libraries were constructed using Chromium Single Cell 3′ Reagent Kit (v3) according to the manufacturer's protocol. Amplified cDNA and final libraries were evaluated on an Agilent Bioanalyzer using a High Sensitivity DNA Kit (Agilent Technologies). Individual libraries were diluted and pooled for sequencing on the NovaSeq 6000 Sequencing System (Illumina) with a target of 350 million reads.

Single-cell RNA sequencing analysis. Sequencing results were demultiplexed and converted to FASTQ format using Illumina bcl2fastq software. The Cell Ranger pipeline (10× Genomics) was used to perform sample demultiplexing, barcode processing, and single-cell 3′ gene counting. Transcripts were aligned to a custom mm10/GRCm38 reference genome that included the sequences of GFP and tdTomato. Additional processing was performed using the Seurat R package. Low-quality barcodes were filtered out according to the cell quality metrics: total UMI counts, genes detected, and mitochondrial proportion. The filtration process began by applying a minimum cutoff of 700 genes detected and a minimum cutoff of 1000 total UMI counts. After these cutoffs were applied, barcodes were further excluded by removing those at the extreme ends of the distributions of the three quality metrics. This process was completed separately for each sample.

To prepare for clustering and differential expression analysis, counts were normalized with the R package sctransform 73. Genes used for clustering were selected by identifying genes within each sample that were highly variable and were expressed at a level above what is typical for the proportion of cells they were detected in. The intersection of the sets of genes identified within each sample was then taken as the final set to use for clustering.

Clustering was performed using Seurat's CCA 74 based integration protocol to integrate the data with “animal” as the batch factor. Differential expression was performed in Seurat using Wilcoxon Rank Sum tests. Tests were conducted between each cluster and all others as well as between each pair of clusters at a series of different clustering resolutions.

To assess the contribution of the disease cells to each cluster, binomial models were fit to the data using a generalized linear model with membership in a cluster as the response variable and either “condition” or “animal” included as a predictive factor. The significance of the effect of the disease state or disease animal was assessed using Wald tests implemented in the R package aod (Lesnoff, M., Lancelot, R. (2012). aod: Analysis of Overdispersed Data. R package version 1.3.1, URL http://cran.r-project.org/package=aod).

Single-cell trajectory analysis. The MSC population included for analysis was generated by excluding all endothelial cells, glial cells, and actively cycling cells from the original dataset. Monocle 3 was applied to construct a single-cell trajectory for these cells. The trajectory was then projected to the same UMAP embedding generated by Seurat during cell clustering analysis. The root of the trajectory was determined by scVelo 75, which characterizes the transcriptional dynamics of splicing kinetics (i.e. RNA velocity) with an unsupervised likelihood-based dynamical model. The gene expression pattern across pseudotime was plotted by the plot_genes_in_pseudotime utility in Monocle 3. Marker genes specifically expressed by each cell type were determined by top_markers function in Monocle 3, with marker genes for a given cell type ranked as top 3 according to ‘pseudo_R2’ among all genes and expressed in a least 10% of cells.

Genes that change as function of pseudotime within a lineage were identified by graph_test function in Monocle 3, with ‘neighbor_graph’ parameter set as “principal_graph”. Functional enrichment analysis was performed using DAVID on genes that significantly changed across pseudotime (q_value<0.05) and showed similar expression patterns among neighboring cells for each lineage.

Cell-cell communication analysis. Cell-cell communications were explored among all cell types using CellChat 48. For cell-cell communication analysis corresponding to each condition, a CellChat object was constructed for control and PMF, respectively. For conditional cell-cell communication analysis, a combined CellChat object was constructed by merging the control and PMF objects together.

The aggregated interaction numbers and weights between different cell types in control and PMF were generated as two circle plots using ‘netVisual_circle’ function in CellChat, with edge weights scaled to be comparable between control and PMF. The major sources and targets for all cell-cell communications within a cell population were generated as a scatter plot using an adapted version of ‘netAnalysis_signalingRole_scatter’ function in CellChat. The communication network in a given signaling pathway was characterized by a hierarchy plot generated by ‘netVisual_aggregate’ function in CellChat. Contribution of each ligand-receptor pair to the overall signaling pathway was visualized by ‘netAnalysis_contribution’ function in CellChat. The gene expression distribution of signaling genes related to the Notch pathway was visualized using a Seurat wrapper function ‘plotGeneExpression’.

The conserved and context-specific signaling pathways between PMF and control were identified and visualized using ‘rankNet’ method in CellChat. A comparison of the outgoing (or incoming) signaling patterns between control and PMF cells was carried out and visualized using ‘netAnalysis_signalingRole_heatmap’. Differential interaction numbers among cell types between control and PMF were shown as a heatmap using ‘netVisual_heatmap’ on the combined CellChat object.

### Quantification and Statistical Analysis

All statistical analysis with the exception of the scRNA-seq analysis was conducted using the Prism software (GraphPad). Results are presented as mean values±standard deviation (SD) or mean values±standard error of the mean (SEM). Quantitative data were evaluated using unpaired Student's t-test or one-way ANOVA. A p value of <0.05 was considered significant. No statistical method was used to predetermine sample sizes. Sample size was determined based on previous literature and experience with the given model systems to ensure accurate assessment of potential biologically important differences.

### Use Examples

In an example, a subject who has, or whom has been diagnosed with, a myelofibrosis is administered a pharmaceutical composition comprising an agent that inhibits proliferation of Sox10+ glial cells in a subject, which agent comprises 6-hydroxydopamine. After one or more doses of the pharmaceutical composition comprising 6-hydroxydopamine are administered, the patient exhibits one or more improvements in symptom(s) of myelofibrosis.

In an example, a subject who has, or whom has been diagnosed with, a myelofibrosis is administered a pharmaceutical composition comprising an agent that inhibits proliferation of ErbB3+ glial cells in a subject, which agent comprises an antibody or antibody fragment agent which blocks ErbB3. After one or more doses of the pharmaceutical composition are administered, the patient exhibits one or more improvements in symptom(s) of myelofibrosis.

In an example, a Jak inhibitor is being used for treating myelofibrosis with disappointing results. The subject is treated with a combined Jak inhibition and ErbB3 pathway inhibition, using, e.g., an antibody or antibody fragment agent which blocks ErbB3. After one or more doses of the combined treatment, the patient exhibits one or more improvements in symptom(s) of myelofibrosis. The combined treatment targets both the hematopoietic compartment and the bone marrow niche.

### REFERENCES

1. Tefferi, A. (2016). Myeloproliferative neoplasms: A decade of discoveries and treatment advances. American journal of hematology 91, 50-58. 10.1002/ajh.24221.
2. Villeval, J. L., Cohen-Solal, K., Tulliez, M., Giraudier, S., Guichard, J., Burstein, S. A., Cramer, E. M., Vainchenker, W., and Wendling, F. (1997). High thrombopoietin production by hematopoietic cells induces a fatal myeloproliferative syndrome in mice. Blood 90, 4369-4383.
3. Pikman, Y., Lee, B. H., Mercher, T., McDowell, E., Ebert, B. L., Gozo, M., Cuker, A., Wernig, G., Moore, S., Galinsky, I., et al. (2006). MPLW515L is a novel somatic activating mutation in myelofibrosis with myeloid metaplasia. PLoS Med 3, e270. 10.1371/journal.pmed.0030270.
4. Li, J., Kent, D. G., Chen, E., and Green, A. R. (2011). Mouse models of myeloproliferative neoplasms: JAK of all grades. Dis Model Mech 4, 311-317. 10.1242/dmm.006817.
5. Yan, X. Q., Lacey, D., Fletcher, F., Hartley, C., McElroy, P., Sun, Y., Xia, M., Mu, S., Saris, C., Hill, D., et al. (1995). Chronic exposure to retroviral vector encoded MGDF (mpl-ligand) induces lineage-specific growth and differentiation of megakaryocytes in mice. Blood 86, 4025-4033.
6. Vainchenker, W., and Kralovics, R. (2017). Genetic basis and molecular pathophysiology of classical myeloproliferative neoplasms. Blood 129, 667-679. 10.1182/blood-2016-10-695940.
7. Ding, L., Saunders, T. L., Enikolopov, G., and Morrison, S. J. (2012). Endothelial and perivascular cells maintain haematopoietic stem cells. Nature 481, 457-462. 10.1038/nature10783.
8. Ding, L., and Morrison, S. J. (2013). Haematopoietic stem cells and early lymphoid progenitors occupy distinct bone marrow niches. Nature 495, 231-235. 10.1038/nature11885.
9. Greenbaum, A., Hsu, Y. M., Day, R. B., Schuettpelz, L. G., Christopher, M. J., Borgerding, J. N., Nagasawa, T., and Link, D. C. (2013). CXCL12 in early mesenchymal progenitors is required for haematopoietic stem-cell maintenance. Nature 495, 227-230. 10.1038/nature11926.
10. Omatsu, Y., Sugiyama, T., Kohara, H., Kondoh, G., Fujii, N., Kohno, K., and Nagasawa, T. (2010). The essential functions of adipo-osteogenic progenitors as the hematopoietic stem and progenitor cell niche. Immunity 33, 387-399. 10.1016/j.immuni.2010.08.017.
11. Zhou, B. O., Yue, R., Murphy, M. M., Peyer, J. G., and Morrison, S. J. (2014). Leptin-receptor-expressing mesenchymal stromal cells represent the main source of bone formed by adult bone marrow. Cell stem cell 15, 154-168. 10.1016/j.stem.2014.06.008.
12. Seike, M., Omatsu, Y., Watanabe, H., Kondoh, G., and Nagasawa, T. (2018). Stem cell niche-specific Ebf3 maintains the bone marrow cavity. Genes & development 32, 359-372. 10.1101/gad.311068.117.
13. Yamazaki, S., Ema, H., Karlsson, G., Yamaguchi, T., Miyoshi, H., Shioda, S., Taketo, M. M., Karlsson, S., Iwama, A., and Nakauchi, H. (2011). Nonmyelinating Schwann cells maintain hematopoietic stem cell hibernation in the bone marrow niche. Cell 147, 1146-1158. 10.1016/j.cell.2011.09.053.
14. Kunisaki, Y., Bruns, I., Scheiermann, C., Ahmed, J., Pinho, S., Zhang, D., Mizoguchi, T., Wei, Q., Lucas, D., Ito, K., et al. (2013). Arteriolar niches maintain haematopoietic stem cell quiescence. Nature 502, 637-643. 10.1038/nature12612.
15. Baryawno, N., Przybylski, D., Kowalczyk, M. S., Kfoury, Y., Severe, N., Gustafsson, K., Kokkaliaris, K. D., Mercier, F., Tabaka, M., Hofree, M., et al. (2019). A Cellular Taxonomy of the Bone Marrow Stroma in Homeostasis and Leukemia. Cell 177, 1915-1932 e1916. 10.1016/j.cell.2019.04.040.
16. Tikhonova, A. N., Dolgalev, I., Hu, H., Sivaraj, K. K., Hoxha, E., Cuesta-Dominguez, A., Pinho, S., Akhmetzyanova, I., Gao, J., Witkowski, M., et al. (2019). The bone marrow microenvironment at single-cell resolution. Nature 569, 222-228. 10.1038/s41586-019-1104-8.
17. Baccin, C., Al-Sabah, J., Velten, L., Helbling, P. M., Grunschlager, F., Hernandez-Malmierca, P., Nombela-Arrieta, C., Steinmetz, L. M., Trumpp, A., and Haas, S. (2020). Combined single-cell and spatial transcriptomics reveal the molecular, cellular and spatial bone marrow niche organization. Nat Cell Biol 22, 38-48. 10.1038/s41556-019-0439-6.
18. Decker, M., Martinez-Morentin, L., Wang, G., Lee, Y., Liu, Q., Leslie, J., and Ding, L. (2017). Leptin-receptor-expressing bone marrow stromal cells are myofibroblasts in primary myelofibrosis. Nat Cell Biol 19, 677-688. 10.1038/ncb3530.
19. Schneider, R. K., Mullally, A., Dugourd, A., Peisker, F., Hoogenboezem, R., Van Strien, P. M. H., Bindels, E. M., Heckl, D., Busche, G., Fleck, D., et al. (2017). Gli1(+) Mesenchymal Stromal Cells Are a Key Driver of Bone Marrow Fibrosis and an Important Cellular Therapeutic Target. Cell stem cell 20, 785-800 e788. 10.1016/j.stem.2017.03.008.
20. Mak, K. K., Bi, Y., Wan, C., Chuang, P. T., Clemens, T., Young, M., and Yang, Y. (2008). Hedgehog signaling in mature osteoblasts regulates bone formation and resorption by controlling PTHrP and RANKL expression. Developmental cell 14, 674-688. 10.1016/j.devcel.2008.02.003.
21. Maeda, Y., Nakamura, E., Nguyen, M. T., Suva, L. J., Swain, F. L., Razzaque, M. S., Mackem, S., and Lanske, B. (2007). Indian Hedgehog produced by postnatal chondrocytes is essential for maintaining a growth plate and trabecular bone. Proceedings of the National Academy of Sciences of the United States of America 104, 6382-6387. 10.1073/pnas.0608449104.
22. Mizuhashi, K., Ono, W., Matsushita, Y., Sakagami, N., Takahashi, A., Saunders, T. L., Nagasawa, T., Kronenberg, H. M., and Ono, N. (2018). Resting zone of the growth plate houses a unique class of skeletal stem cells. Nature 563, 254-258. 10.1038/s41586-018-0662-5.
23. Long, F., Chung, U. I., Ohba, S., McMahon, J., Kronenberg, H. M., and McMahon, A. P. (2004). Ihh signaling is directly required for the osteoblast lineage in the endochondral skeleton. Development 131, 1309-1318. 10.1242/dev.01006.
24. Hamilton, T. G., Klinghoffer, R. A., Corrin, P. D., and Soriano, P. (2003). Evolutionary divergence of platelet-derived growth factor alpha receptor signaling mechanisms. Mol Cell Biol 23, 4013-4025. 10.1128/mcb.23.11.4013-4025.2003.
25. Yata, Y., Scanga, A., Gillan, A., Yang, L., Reif, S., Breindl, M., Brenner, D. A., and Rippe, R. A. (2003). DNase I-hypersensitive sites enhance alpha1(I) collagen gene expression in hepatic stellate cells. Hepatology 37, 267-276. 10.1053/jhep.2003.50067.
26. He, L., Huang, X., Kanisicak, O., Li, Y., Wang, Y., Li, Y., Pu, W., Liu, Q., Zhang, H., Tian, X., et al. (2017). Preexisting endothelial cells mediate cardiac neovascularization after injury. The Journal of clinical investigation 127, 2968-2981. 10.1172/JCI93868.
27. Bai, C. B., Auerbach, W., Lee, J. S., Stephen, D., and Joyner, A. L. (2002). Gli2, but not Gli1, is required for initial Shh signaling and ectopic activation of the Shh pathway. Development 129, 4753-4761.
28. Merchant, A., Joseph, G., Wang, Q., Brennan, S., and Matsui, W. (2010). Gli1 regulates the proliferation and differentiation of HSCs and myeloid progenitors. Blood 115, 2391-2396. 10.1182/blood-2009-09-241703.
29. Gao, J., Graves, S., Koch, U., Liu, S., Jankovic, V., Buonamici, S., El Andaloussi, A., Nimer, S. D., Kee, B. L., Taichman, R., et al. (2009). Hedgehog signaling is dispensable for adult hematopoietic stem cell function. Cell stem cell 4, 548-558. 10.1016/j.stem.2009.03.015.
30. Tibes, R., and Mesa, R. A. (2014). Targeting hedgehog signaling in myelofibrosis and other hematologic malignancies. J Hematol Oncol 7, 18. 10.1186/1756-8722-7-18.
31. Kleppe, M., Koche, R., Zou, L., van Galen, P., Hill, C. E., Dong, L., De Groote, S., Papalexi, E., Hanasoge Somasundara, A. V., Cordner, K., et al. (2018). Dual Targeting of Oncogenic Activation and Inflammatory Signaling Increases Therapeutic Efficacy in Myeloproliferative Neoplasms. Cancer cell 33, 29-43 e27. 10.1016/j.ccell.2017.11.009.
32. Rauch, A., Haakonsson, A. K., Madsen, J. G. S., Larsen, M., Forss, I., Madsen, M. R., Van Hauwaert, E. L., Wiwie, C., Jespersen, N. Z., Tencerova, M., et al. (2019). Osteogenesis depends on commissioning of a network of stem cell transcription factors that act as repressors of adipogenesis. Nature genetics 51, 716-727. 10.1038/s41588-019-0359-1.
33. Acar, M., Kocherlakota, K. S., Murphy, M. M., Peyer, J. G., Oguro, H., Inra, C. N., Jaiyeola, C., Zhao, Z., Luby-Phelps, K., and Morrison, S. J. (2015). Deep imaging of bone marrow shows non-dividing stem cells are mainly perisinusoidal. Nature 526, 126-130. 10.1038/nature15250.
34. Morrison, S. J., and Scadden, D. T. (2014). The bone marrow niche for haematopoietic stem cells. Nature 505, 327-334. 10.1038/nature12984.
35. Lee, Y., Decker, M., Lee, H., and Ding, L. (2017). Extrinsic regulation of hematopoietic stem cells in development, homeostasis and diseases. Wiley Interdiscip Rev Dev Biol 6. 10.1002/wdev.279.
36. Kusumbe, A. P., Ramasamy, S. K., and Adams, R. H. (2014). Coupling of angiogenesis and osteogenesis by a specific vessel subtype in bone. Nature 507, 323-328. 10.1038/nature13145.
37. Mesa, R. A., Hanson, C. A., Rajkumar, S. V., Schroeder, G., and Tefferi, A. (2000). Evaluation and clinical correlations of bone marrow angiogenesis in myelofibrosis with myeloid metaplasia. Blood 96, 3374-3380.
38. Panteli, K., Zagorianakou, N., Bai, M., Katsaraki, A., Agnantis, N.J., and Bourantas, K. (2004). Angiogenesis in chronic myeloproliferative diseases detected by CD34 expression. European journal of haematology 72, 410-415. 10.1111/j.1600-0609.2004.00235.x.
39. Boveri, E., Passamonti, F., Rumi, E., Pietra, D., Elena, C., Arcaini, L., Pascutto, C., Castello, A., Cazzola, M., Magrini, U., and Lazzarino, M. (2008). Bone marrow microvessel density in chronic myeloproliferative disorders: a study of 115 patients with clinicopathological and molecular correlations. British journal of haematology 140, 162-168. 10.1111/j.1365-2141.2007.06885.x.
40. Ramasamy, S. K., Kusumbe, A. P., Wang, L., and Adams, R. H. (2014). Endothelial Notch activity promotes angiogenesis and osteogenesis in bone. Nature 507, 376-380. 10.1038/nature13146.
41. Shih, C. H., Lacagnina, M., Leuer-Bisciotti, K., and Proschel, C. (2014). Astroglial-derived periostin promotes axonal regeneration after spinal cord injury. J Neurosci 34, 2438-2443. 10.1523/JNEUROSCI.2947-13.2014.
42. Ma, S. M., Chen, L. X., Lin, Y. F., Yan, H., Lv, J. W., Xiong, M., Li, J., Cheng, G. Q., Yang, Y., Qiu, Z. L., and Zhou, W. H. (2015). Periostin Promotes Neural Stem Cell Proliferation and Differentiation following Hypoxic-Ischemic Injury. PloS one 10, e0123585. 10.1371/journal.pone.0123585.
43. Lyons, D. A., Pogoda, H. M., Voas, M. G., Woods, I. G., Diamond, B., Nix, R., Arana, N., Jacobs, J., and Talbot, W. S. (2005). erbb3 and erbb2 are essential for schwann cell migration and myelination in zebrafish. Curr Biol 15, 513-524. 10.1016/j.cub.2005.02.030.
44. Brinkmann, B. G., Agarwal, A., Sereda, M. W., Garratt, A. N., Muller, T., Wende, H., Stassart, R. M., Nawaz, S., Humml, C., Velanac, V., et al. (2008). Neuregulin-1/ErbB signaling serves distinct functions in myelination of the peripheral and central nervous system. Neuron 59, 581-595. 10.1016/j.neuron.2008.06.028.
45. Riethmacher, D., Sonnenberg-Riethmacher, E., Brinkmann, V., Yamaai, T., Lewin, G. R., and Birchmeier, C. (1997). Severe neuropathies in mice with targeted mutations in the ErbB3 receptor. Nature 389, 725-730. 10.1038/39593.
46. McKenzie, I. A., Ohayon, D., Li, H., de Faria, J. P., Emery, B., Tohyama, K., and Richardson, W. D. (2014). Motor skill learning requires active central myelination. Science 346, 318-322. 10.1126/science.1254960.
47. Chagraoui, H., Komura, E., Tulliez, M., Giraudier, S., Vainchenker, W., and Wendling, F. (2002). Prominent role of TGF-beta 1 in thrombopoietin-induced myelofibrosis in mice. Blood 100, 3495-3503. 10.1182/blood-2002-04-1133.
48. Jin, S., Guerrero-Juarez, C. F., Zhang, L., Chang, I., Ramos, R., Kuan, C. H., Myung, P., Plikus, M. V., and Nie, Q. (2021). Inference and analysis of cell-cell communication using CellChat. Nature communications 12, 1088. 10.1038/s41467-021-21246-9.
49. Hilton, M. J., Tu, X., Wu, X., Bai, S., Zhao, H., Kobayashi, T., Kronenberg, H. M., Teitelbaum, S. L., Ross, F. P., Kopan, R., and Long, F. (2008). Notch signaling maintains bone marrow mesenchymal progenitors by suppressing osteoblast differentiation. Nature medicine 14, 306-314. 10.1038/nm1716.
50. Engin, F., Yao, Z., Yang, T., Zhou, G., Bertin, T., Jiang, M. M., Chen, Y., Wang, L., Zheng, H., Sutton, R. E., et al. (2008). Dimorphic effects of Notch signaling in bone homeostasis. Nature medicine 14, 299-305. 10.1038/nm1712.
51. Rameshwar, P., Chang, V. T., Thacker, U. F., and Gascon, P. (1998). Systemic transforming growth factor-beta in patients with bone marrow fibrosis-pathophysiological implications. American journal of hematology 59, 133-142. 10.1002/(sici)1096-8652(199810)59:2<133::aid-ajh6>3.0.co; 2-z.
52. Yanagida, M., Ide, Y., Imai, A., Toriyama, M., Aoki, T., Harada, K., Izumi, H., Uzumaki, H., Kusaka, M., and Tokiwa, T. (1997). The role of transforming growth factor-beta in PEG-rHuMGDF-induced reversible myelofibrosis in rats. British journal of haematology 99, 739-745. 10.1046/j.1365-2141.1997.4843288.x.
53. Schmitt, A., Jouault, H., Guichard, J., Wendling, F., Drouin, A., and Cramer, E. M. (2000). Pathologic interaction between megakaryocytes and polymorphonuclear leukocytes in myelofibrosis. Blood 96, 1342-1347.
54. Kvist-Reimer, M., Sundler, F., and Ahren, B. (2002). Effects of chemical sympathectomy by means of 6-hydroxydopamine on insulin secretion and islet morphology in alloxan-diabetic mice. Cell Tissue Res 307, 203-209. 10.1007/s00441-001-0496-5.
55. Angeletti, P. U., and Levi-Montalcini, R. (1970). Sympathetic nerve cell destruction in newborn mammals by 6-hydroxydopamine. Proceedings of the National Academy of Sciences of the United States of America 65, 114-121. 10.1073/pnas.65.1.114.
56. Tsunokuma, N., Yamane, T., Matsumoto, C., Tsuneto, M., Isono, K., Imanaka-Yoshida, K., and Yamazaki, H. (2017). Depletion of Neural Crest-Derived Cells Leads to Reduction in Plasma Noradrenaline and Alters B Lymphopoiesis. J Immunol 198, 156-169. 10.4049/jimmunol.1502592.
57. Katayama, Y., Battista, M., Kao, W. M., Hidalgo, A., Peired, A. J., Thomas, S. A., and Frenette, P. S. (2006). Signals from the sympathetic nervous system regulate hematopoietic stem cell egress from bone marrow. Cell 124, 407-421. 10.1016/j.cell.2005.10.041.
58. Kuppe, C., Ibrahim, M. M., Kranz, J., Zhang, X., Ziegler, S., Perales-Paton, J., Jansen, J., Reimer, K. C., Smith, J. R., Dobie, R., et al. (2021). Decoding myofibroblast origins in human kidney fibrosis. Nature 589, 281-286. 10.1038/s41586-020-2941-1.
59. Shi, Y., He, G., Lee, W. C., McKenzie, J. A., Silva, M. J., and Long, F. (2017). Gli1 identifies osteogenic progenitors for bone formation and fracture repair. Nature communications 8, 2043. 10.1038/s41467-017-02171-2.
60. Gerds, A. T., Tauchi, T., Ritchie, E., Deininger, M., Jamieson, C., Mesa, R., Heaney, M., Komatsu, N., Minami, H., Su, Y., et al. (2019). Phase 1/2 trial of glasdegib in patients with primary or secondary myelofibrosis previously treated with ruxolitinib. Leuk Res 79, 38-44. 10.1016/j.leukres.2019.02.012.
61. Couban, S., Benevolo, G., Donnellan, W., Cultrera, J., Koschmieder, S., Verstovsek, S., Hooper, G., Hertig, C., Tandon, M., Dimier, N., et al. (2018). A phase Ib study to assess the efficacy and safety of vismodegib in combination with ruxolitinib in patients with intermediate- or high-risk myelofibrosis. J Hematol Oncol 11, 122. 10.1186/s13045-018-0661-x.
62. Sasaki, K., Gotlib, J. R., Mesa, R. A., Newberry, K. J., Ravandi, F., Cortes, J. E., Kelly, P., Kutok, J. L., Kantarjian, H. M., and Verstovsek, S. (2015). Phase II evaluation of IPI-926, an oral Hedgehog inhibitor, in patients with myelofibrosis. Leuk Lymphoma 56, 2092-2097. 10.3109/10428194.2014.984703.
63. Leimkuhler, N. B., Gleitz, H. F. E., Ronghui, L., Snoeren, I. A. M., Fuchs, S. N. R., Nagai, J. S., Banjanin, B., Lam, K. H., Vogl, T., Kuppe, C., et al. (2021). Heterogeneous bone-marrow stromal progenitors drive myelofibrosis via a druggable alarmin axis. Cell stem cell 28, 637-652 e638. 10.1016/j.stem.2020.11.004.
64. Yang, S., Corbett, S. E., Koga, Y., Wang, Z., Johnson, W. E., Yajima, M., and Campbell, J. D. (2020). Decontamination of ambient RNA in single-cell RNA-seq with DecontX. Genome Biol 21, 57. 10.1186/s13059-020-1950-6.
65. Yao, J. C., Oetjen, K. A., Wang, T., Xu, H., Abou-Ezzi, G., Krambs, J. R., Uttarwar, S., Duncavage, E. J., and Link, D. C. (2022). TGF-beta signaling in myeloproliferative neoplasms contributes to myelofibrosis without disrupting the hematopoietic niche. The Journal of clinical investigation 132. 10.1172/JCI154092.
66. Ahn, S., and Joyner, A. L. (2004). Dynamic changes in the response of cells to positive hedgehog signaling during mouse limb patterning. Cell 118, 505-516. 10.1016/j.cell.2004.07.023.
67. Madisen, L., Zwingman, T. A., Sunkin, S. M., Oh, S. W., Zariwala, H. A., Gu, H., Ng, L. L., Palmiter, R. D., Hawrylycz, M. J., Jones, A. R., et al. (2010). A robust and high-throughput Cre reporting and characterization system for the whole mouse brain. Nature neuroscience 13, 133-140. 10.1038/nn.2467.
68. Buch, T., Heppner, F. L., Tertilt, C., Heinen, T. J., Kremer, M., Wunderlich, F. T., Jung, S., and Waisman, A. (2005). A Cre-inducible diphtheria toxin receptor mediates cell lineage ablation after toxin administration. Nature methods 2, 419-426. 10.1038/nmeth762.
69. Lakso, M., Pichel, J. G., Gorman, J. R., Sauer, B., Okamoto, Y., Lee, E., Alt, F. W., and Westphal, H. (1996). Efficient in vivo manipulation of mouse genomic sequences at the zygote stage. Proceedings of the National Academy of Sciences of the United States of America 93, 5860-5865. 10.1073/pnas.93.12.5860.
70. DeFalco, J., Tomishima, M., Liu, H., Zhao, C., Cai, X., Marth, J. D., Enquist, L., and Friedman, J. M. (2001). Virus-assisted mapping of neural inputs to a feeding center in the hypothalamus. Science 291, 2608-2613. 10.1126/science.1056602.
71. Rodriguez, C. I., Buchholz, F., Galloway, J., Sequerra, R., Kasper, J., Ayala, R., Stewart, A. F., and Dymecki, S. M. (2000). High-efficiency deleter mice show that FLPe is an alternative to Cre-loxP. Nature genetics 25, 139-140. 10.1038/75973.
72. Thiele, J., Kvasnicka, H. M., Facchetti, F., Franco, V., van der Walt, J., and Orazi, A. (2005). European consensus on grading bone marrow fibrosis and assessment of cellularity. Haematologica 90, 1128-1132.
73. Hafemeister, C., and Satija, R. (2019). Normalization and variance stabilization of single-cell RNA-seq data using regularized negative binomial regression. Genome Biol 20, 296. 10.1186/s13059-019-1874-1.
74. Stuart, T., Butler, A., Hoffman, P., Hafemeister, C., Papalexi, E., Mauck, W. M., 3rd, Hao, Y., Stoeckius, M., Smibert, P., and Satija, R. (2019). Comprehensive Integration of Single-Cell Data. Cell 177, 1888-1902 e1821. 10.1016/j.cell.2019.05.031.
75. Bergen, V., Lange, M., Peidli, S., Wolf, F. A., and Theis, F. J. (2020). Generalizing RNA velocity to transient cell states through dynamical modeling. Nature Biotechnology 38. 10.1038/s41587-020-0591-3.

1. A method for treating a myelofibrosis in a subject comprising administering to the subject a pharmaceutical composition comprising an agent that depletes, or inhibits proliferation of, Sox10+ glial cells in a subject and/or ErbB3+ glial cells in a subject, so as to thereby treat the myelofibrosis.

2. The method of claim 1, wherein the Sox10+ glial cells are not surgically ablated from the subject.

3. The method of claim 1, wherein the agent is a small molecule.

4. The method of claim 1, wherein the agent comprises 6-hydroxydopamine.

5. The method of claim 1, wherein the pharmaceutical composition comprises a conjugate of 6-hydroxydopamine.

6. The method of claim 1, wherein the agent is a cytotoxic agent which targets ErbB3 or an agent which blocks ErbB3.

7. The method of claim 6, wherein the agent comprises an anti-ErbB3 antibody or ErbB3-binding fragment thereof.

8. The method of claim 1, wherein the method does not evoke body weight loss in the subject.

9. The method of claim 1, wherein the agent selectively depletes, or selectively inhibits proliferation of, Sox10+ glial cells over other glial cell types.

10. The method of claim 1, wherein the agent does not cross a blood-brain barrier in the subject.

11. The method of claim 1, wherein the method effects depletion of, or inhibition of proliferation of, Sox10+ glial cells in bone marrow of the subject.

12. The method of claim 1, further comprising diagnosing the subject, or having the subject diagnosed, as having a myelofibrosis prior to treatment.

13. The method of claim 12, wherein the subject is diagnosed on the basis of a bone marrow biopsy.

14. (canceled)

15. (canceled)

16. The method of claim 1, wherein the agent is not a Janus kinase (JAK) inhibitor.

17. (canceled)

18. (canceled)

19. (canceled)

20. (canceled)

21. The method of claim 1, wherein the agent comprises AZD8931.

22. The method of claim 21, wherein a JAK inhibitor is additionally administered to the subject.

23. The method of claim 7, wherein the anti-ErbB3 antibody is Elgemtumab (LJM716), Lumretuzumab (RG7116) or KTN3379.

24. The method of claim 22, wherein the JAK inhibitor is abrocitinib, baricitinib, delgocitinib, fedratinib, filgotinib, oclacitinib, pacritinib, peficitinib, ruxolitinib, tofacitinib, and upadacitinib.

25. A bone marrow-targeting therapeutic composition comprising (i) an agent that depletes, or inhibits proliferation of, Sox10+ glial cells in a subject and/or ErbB3+ glial cells and (ii) a molecular entity that targets bone marrow.

26. (canceled)

27. (canceled)

28. (canceled)

29. (canceled)

30. (canceled)

31. (canceled)

32. (canceled)

33. (canceled)

34. A method for treating a myelofibrosis in a subject comprising administering to the subject an agent that depletes, or inhibits proliferation of, ErbB3+ glial cells in a subject, and a Janus kinase inhibitor, so as to thereby treat the myelofibrosis.
